(19) United States
(12) Patent Application Publication
(10) Pub. No.: US 2013/0108645 A1
(43) Pub. Date: May 2, 2013

(54) METHODS FOR ENHANCING AXONAL REGENERATION

(71) Applicant: Mohamed H. Farah, Cockeysville, MD (US)

(72) Inventor: Mohamed H. Farah, Cockeysville, MD (US)

(73) Assignee: THE JOHNS HOPKINS UNIVERSITY, Baltimore, MD (US)

(21) Appl. No.: 13/640,984

(22) PCT Filed: Apr. 13, 2011
(86) PCT No.: PCT/US2011/032219

§ 371 Date: Dec. 27, 2012

Related U.S. Application Data
(60) Provisional application No. 61/323,630, filed on Apr. 13, 2010.

(51) Int. Cl. A61K 31/7088; A61K 39/395

(52) U.S. Cl. CPC A61K 31/7088; A61K 39/3955
USPC 424/158.1; 435/375; 514/44 A; 424/172.1; 435/7.1; 435/7.9; 435/40.52

(57) ABSTRACT

The invention provides novel methods of enhancing axonal regeneration. The methods of the invention are suitable for use in treating a subject suffering from a nerve injury.

### CROSS-REFERENCE TO RELATED APPLICATION

This application claims the benefit of U.S. Provisional Application No. 61/323,630, filed Apr. 13, 2010, the contents of which is incorporated herein by reference in its entirety.

### STATEMENT OF RIGHTS TO INVENTIONS MADE UNDER FEDERALLY SPONSORED RESEARCH

This work was supported by grant no. R01 NS041269 from the National Institutes of Neurological Disease and Stroke of the National Institutes of Health, and Adelson Program in Neural Repair and Rehabilitation. The U.S. Government has certain rights in this invention.

### BACKGROUND OF THE INVENTION

Damage to the nervous system, both central (CNS) and peripheral (PNS), can result from several causes including physical injury, ischemia, neurological disorders, certain medical procedures or therapies, tumors, infections, metabolic or nutritional disorders, cognition or mood disorders, and various diseases. Recovery from such injuries is typically poor because the injured nervous system is an inhibitory environment for axon regeneration that severely limits functional recovery. Despite intensive research, there remains a need to develop effective methods that can promote significant nerve regeneration after nerve injury.

In humans, axonal regeneration after nerve injury is slow and often functionally incomplete. Axonal regeneration is influenced by the intrinsic growth state of local axonal protein synthesis, cytoskeletal organization, growth factors, extracellular matrix, and the clearance of myelin debris from the injured nerve. In turn, these factors variably influence the latency period before initiation of axonal growth, rate of axonal outgrowth, specificity of target reinnervation, and the speed of recovery. In damaged human nerves that require long distance regeneration, shortening the latency period is unlikely to substantially contribute to faster recovery. However, manipulation of molecular pathways that speed the rate of axonal regeneration would be a highly desirable therapeutic approach. Various signaling pathways have been suggested to improve axonal regeneration in the nervous system, but no molecular or pharmacological therapy demonstrating efficacy exists for injured nerves in human.

β-site amyloid precursor protein cleaving enzyme type 1 (BACE1) is a trans-membrane aspartyl protease that is enriched in the developing nervous system, particularly in spinal cord and dorsal root ganglia. BACE1 is expressed in adult neurons, including axons. BACE1 cleaves neuregulin 1 type III, a protein critical for myelination and proper segregation of peripheral axons during development. BACE1 knockout mice exhibit delayed myelination, hypomyelination of large diameter axons, and aberrant Remak bundles containing large numbers of small unmyelinated axons in the sciatic nerve. After a nerve crush, delay- and hyomyelination reemerge in BACE1 KO nerve, which likely result from reduced cleavage and signaling of neuregulin 1 type III.

In addition, BACE1 cleaves amyloid precursor protein (APP), which is a protein implicated in the development of Alzheimer's Disease. BACE1 cleaves APP to generate a soluble amino-terminal fragment, N-APP, and a carboxyl-terminal fragment that is further processed by the γ-secretase complex to generate amyloid-β peptides. It has previously been shown that reduction of APP by genetic deletion and by RNA interference increases neurite outgrowth. In addition, N-APP has been reported to induce axonal degeneration during withdrawal of nerve growth factor from cultured embryonic neurons. However, it remains unclear whether APP is involved in axonal health as it has also been shown that increasing soluble APP increases neurite outgrowth. Accordingly, further studies are needed to determine whether BACE1 is a molecular target for treating injured nerves.

### SUMMARY OF THE INVENTION

As described below, this invention provides novel methods and compositions for increasing nerve regeneration in a subject.

In one aspect, the invention provides methods for enhancing axonal outgrowth. In embodiments, the methods involve contacting an axon with an effective amount of an agent that reduces the expression or biological activity of BACE1.

In another aspect, the invention provides methods for enhancing axonal regeneration. In embodiments, the methods involve contacting an axon with an effective amount of an agent that reduces the expression or biological activity of BACE1.

In another aspect, the invention provides methods for enhancing reinnervation of a target tissue. In embodiments, the methods involve contacting a neuron and/or the target tissue with an effective amount of an agent that reduces the expression or biological activity of BACE1.

In the above aspects, the methods are carried out in vitro or in vivo. In embodiments, the methods accelerate axonal outgrowth. In embodiments, the axon is a motor neuron axon.

In another aspect, the invention provides methods for preventing, treating, and/or reducing symptoms of nerve injury in a subject. In embodiments, the methods involve administering to the subject an effective amount of an agent that reduces the expression or biological activity of BACE1, thereby preventing, treating, and/or reducing symptoms of nerve injury in the subject. In embodiments, the methods involve administering the agent in an amount sufficient to increase nerve regeneration in the subject. In embodiments, the methods involve administering the agent in an amount sufficient to re-innervate nueromuscular junctions, increase axon regeneration, or increase the formation of new presynaptic terminals in the subject. In embodiments, methods involve administering the agent in an amount sufficient to increase clearance of axon and myelin debris in the subject. In embodiments, the methods enhance motor or sensory function in the subject.

In another aspect, the invention provides methods for increasing axonal regeneration in a subject. In embodiments, the methods involve administering to the subject an effective amount of an agent that inhibits the expression or biological activity of (BACE1), thereby increasing axonal regeneration. In embodiments, the methods involve administering the agent in an amount sufficient to increase nerve regeneration in the subject. In embodiments, the methods involve administering the agent in an amount sufficient to re-innervate nueromuscular junctions, increase axon regeneration, or increase the formation of new presynaptic terminals in the subject. In embodiments, methods involve administering the agent in an amount sufficient to increase clearance of axon and myelin debris in the subject. In embodiments, the methods enhance motor or sensory function in the subject.

In any of the above aspects, the nerve injury can be a peripheral nervous system injury.

In any of the above aspects, the nerve injury can be a central nervous system injury.

In any of the above aspects, the nerve injury can be a neuropathy characterized by degeneration of axons. In embodiments, the neuropathy is diabetic neuropathy. In embodiments, the neuropathy is chemotherapy-induced neuropathy.

In any of the above aspects, the subject is a mammal. In embodiments, the subject is human. In related embodiments, the subject is identified as having nerve injury, including, but not limited to, traumatic brain injury, spinal cord injury, ischemic injury, and injury associated with neurological disorder. In embodiments, the subject is identified as having muscular dystrophy.

In any of the above aspects, the agent can be a peptide, polypeptide, polynucleotide, or small molecule. In embodiments, the agent is an inhibitory nucleic acid molecule. In related embodiments, the inhibitory nucleic acid molecule is an antisense nucleic acid molecule, an siRNA, or an shRNA that reduces the expression of BACE1. In embodiments, the agent is an antibody that specifically binds to BACE1 or inhibits BACE1 cleavage of amyloid precursor protein. In embodiments, the agent is a peptidomimetic inhibitor of BACE1, a non-peptidomimetic inhibitor of BACE1, a macrocyclic inhibitor of BACE1, BACE1 inhibitor IV, OM99-2, OM00-3, GT-1017, KMI-429, KMI-570, KMI-684, CTS-2116, WAY-258131, AZ29, or a pharmaceutically acceptable salt or prodrug thereof. In related embodiments, the agent is a BACE1 inhibitor from Johnson & Johnson, Eisai, Merck, Schering-Plough, and TransTech Pharma. In related embodiments, the inhibitor is BACE1 inhibitor IV.

In any of the above aspects, the agent is administered for a time sufficient to increase axonal regeneration.

In any of the above aspects, the methods enhance motor or sensory function in the subject.

In another aspect, the invention provides methods for increasing nerve regeneration in a mammalian subject after peripheral nerve injury. In embodiments, the methods involve administering to the subject an effective amount of an agent that reduces the expression or biological activity of BACE1. In embodiments, the methods further involve comparing the amount of nerve regeneration in a subject after administration of the agent and modifying the amount and time of administration so that nerve regeneration is increased in the subject as compared to a control.

In embodiments, nerve regeneration is measured by determination of new formation of presynaptic terminals, by determination of enhanced myelin debris removal, and/or by determination of re-innervation of neuromuscular junctions.

In embodiments, the inhibitor is BACE1 inhibitor IV.

In embodiments, the subject is a rodent, bovine, feline, or canine.

In any of the above aspects, the methods further involve administering at least one additional therapeutic molecule. The therapeutic molecule can be any compound used to treat a subject suffering from nerve injury. In embodiments, the therapeutic molecule is any therapeutic molecule described herein. In related embodiments, the therapeutic molecule is Taxol®.

In any of the above aspects, about 50 nM to about 1 μM of the agent and/or therapeutic molecule is administered to the subject.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### Definitions

To facilitate an understanding of the present invention, a number of terms and phrases are defined below.

As used herein, the singular forms “a,” “an,” and “the” include plural forms unless the context clearly dictates otherwise. Thus, for example, reference to “an agent” includes reference to more than one agent.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having,” and the like, can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “BACE1” refers to a β-site amyloid precursor protein cleaving enzyme type 1, which is capable of cleaving amyloid precursor protein (APP) to yield a soluble amino-terminal fragment, N-APP. BACE1 is also known as ASP2 and Memapsin2. Several variants of BACE1 have been sequenced, including variants A, B, C, and D. Full nucleotide sequences encoding human BACE1, and variants related thereto, are well-known in the art (see, e.g., NCBI Accession Nos. NM_{—}138971, NM_{—}138972, NM_{—}138973, and NM_{—}012104, each of which is hereby incorporated by reference).

By “BACE1 nucleic acid molecule” is meant a polynucleotide encoding a BACE1 polypeptide or fragment thereof.

By “BACE1 polypeptide” is meant a protein or fragment thereof, which is capable of cleaving APP, and having at least 85% identity to the amino acid sequence corresponding to NM_{—}138971, NM_{—}138972, NM_{—}138973, or NM_{—}012104.

By “central nervous system” (CNS) is meant the brain or spinal cord, and cellular or molecular components thereof, including the extracellular materials and fluids.

By “central nervous system disease or injury” is meant any disease, disorder, or trauma that disrupts the normal function or connectivity of the brain or spinal cord.

By “peripheral nervous system” (PNS) is meant all other neural elements outside the brain and the spinal cord, including nerves, ganglia, spinal, and cranial nerves.

By “peripheral nervous system disease or injury” is meant any disease, disorder, or trauma that disrupts the normal function of the neural elements outside the brain and spinal cord.

By “agent” is meant any small molecule chemical compound, antibody, nucleic acid molecule, or polypeptide, or fragment thereof.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, or a 50% or greater change in expression levels.

By “control” is meant a standard or reference condition.

By “disease” is meant any condition or disorder that damages or interferes with the normal function of a cell, tissue, or organ.

By “enhances” or “increases” is meant a positive alteration of at least about 10%, 25%, 50%, 75%, or 100% relative to a reference.

By “enhancing axonal outgrowth” is meant increasing the number of axons or the distance of extension of axons relative to a control condition. In embodiments, the increase is by at least 2-fold, 2.5-fold, 3-fold or more.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen, or reversed Hoogsteen hydrogen bonding between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, HPLC analysis, and the like.

By “modifies” is meant alters. In the context of the invention, an agent that modifies a cell, substrate, or cellular environment produces a biochemical alteration in a component (e.g., polypeptide, nucleotide, or molecular component) of the cell, substrate, or cellular environment.

By “neuron” is meant any nerve cell derived from the nervous system of a mammal. The neuron is the basic building block of the nervous system, both CNS and PNS, where it receives, processes and transmits electrical information from one part of the body to another. A neuron consists of a cell body and two or more extensions, called dendrites and axons. Dendrites receive inputs and conduct signals toward the cell body, whereas axons conduct signal away from the body to other neurons or target cells to which they connect. In embodiments, the neuron is a motor neuron.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); and Kimmel, A. R., Methods Enzymol. 152:507 (1987)).

By “reduces” is meant a negative alteration of at least about 10%, 25%, 50%, 75%, or 100% relative to a reference.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides or any integer thereabout or therebetween.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine and tyrosine. In an exemplary approach to determining the degree of identity, a BLAST program may be used, with a probability score between e⁻³ and e⁻¹⁰⁰ indicating a closely related sequence.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “substantially identical” is meant a polypeptide or nucleic acid molecule exhibiting at least 85% identity to a reference amino acid sequence (for example, any one of the amino acid sequences described herein) or nucleic acid sequence (for example, any one of the nucleic acid sequences described herein). Preferably, such a sequence is at least 85%, 90%, 95%, 99% or even 100% identical at the amino acid level or nucleic acid to the sequence used for comparison.

“Administering” is defined herein as a means of providing an agent to a subject in a manner that results in the agent being inside the subject's body. Such an administration can be by any route including, without limitation, oral, transdermal, mucosal (e.g., vagina, rectum, oral, or nasal mucosa), by injection (e.g., subcutaneous, intravenous, parenterally, intraperitoneally, intrathecal), or by inhalation (e.g., oral or nasal). Pharmaceutical preparations are, of course, given by forms suitable for each administration route.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

As used herein, the terms “treat,” treating,” “treatment,” and the like refer to reducing or ameliorating a disorder and/or symptoms associated therewith. By “ameliorate” is meant decrease, suppress, attenuate, diminish, arrest, or stabilize the development or progression of a disease. It will be appreciated that, although not precluded, treating a disorder or condition does not require that the disorder, condition or symptoms associated therewith be completely eliminated.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or adjuvant” refers to an excipient, carrier or adjuvant that can be administered to a subject, together with an agent, and which does not destroy the pharmacological activity thereof and is nontoxic when administered in doses sufficient to deliver a therapeutic amount of the agent.

By “effective amount” is meant the amount of an agent required to ameliorate the symptoms of a disease relative to an untreated patient. The effective amount of an agent used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compounds, compositions, or methods provided herein can be combined with one or more of any of the other compositions and methods provided herein.

### DESCRIPTION OF THE DRAWINGS

FIGS. 1A-1I show the similar degree of axonal and myelin degeneration in WT and BACE1 KO sciatic nerves following axotomy-induced Wallerian degeneration and intoxication with paclitaxel. FIGS. 1A, 1B, 1E, and 1F include electron micrographs and FIGS. 1C, 1D, 1G, and 1H include images of semi-thin (1 μm) plastic sections stained with toluidine blue. FIG. 1A shows uninjured WT axon with normal axoplasm. FIG. 1B shows conversion of the axonal cytoskeleton to granular debris in WT nerve at 48 hours post-crush showing. FIG. 1C shows degeneration of myelin in WT nerve at 5 days post-crush. FIG. 1D shows WT nerve intoxicated with paclitaxel. Arrows point to degenerating myelinated fibers. FIG. 1E shows uninjured BACE1 KO axon with normal axoplasm. FIG. 1F shows conversion of the axonal cytoskeleton to granular debris in BACE1 KO at 48 hr post-crush, similar to WT nerve in FIG. 1B. FIG. 1G shows degeneration of myelin in BACE1 KO nerve at 5 days post-crush. FIG. 1H shows BACE1 KO nerve intoxicated with paclitaxel. Arrows point to degenerating myelinated fibers. Scale bars in FIGS. 1A, 1B, 1D, and 1F=500 nm, and in FIGS. 1C, 1F, 1G, and 1H=100 μm. FIG. 1I is a graph showing quantification of degenerating myelinated fibers in the whole cross-sectional area of nerves of mice intoxicated with paclitaxel. N=3 per genotype. Values are mean±SEM.

FIGS. 2A-2D show enhanced clearance of axonal debris and enhanced axonal regeneration in YFP-BACE1 KO nerves compared to YFP-WT nerves following axotomy. FIGS. 2A-2D include images that are projections of Z-stacks from whole mounted-nerves, showing the YPF-positive axonal debris and YFP-positive regenerated axons through the depth of the nerves. FIG. 2A shows YFP-WT nerve at 7 days post transection. Fragmented YFP debris (arrowhead) is present. FIG. 2B shows YFP-BACE1 KO nerve at 7 days post transection. YFP debris (arrowhead) is largely cleared from the nerve. FIG. 2C shows YFP-WT nerve at 10 days post-crush. Fragmented YFP debris (arrowhead) is present at a segment 10 mm distal to the crush and few regenerated YFP-positive axons (arrow) are growing beneath the debris. FIG. 2D shows YFP-BACE1 KO nerve at 10 days post-crush. Numerous regenerated YFP-positive axons are present at segment 10 mm distal to crush site and (arrows). Scanty clumps of YFP-positive axonal debris also remain. Scale bars=100 μm. Laser scanning intensity was increased in FIGS. 2C and 2D to reveal small diameter YFP-positive regenerated axons.

FIGS. 3A-3I show enhanced phagocytosis by BACE1 KO macrophages both in vivo and in vitro. FIGS. 3A-3F are micrographs of distal stumps at 5 days post-injury. FIG. 3A includes a stain for Iba1 (ionized calcium-binding adaptor molecule 1), a marker for macrophages, and shows that the majority of Iba1-positive macrophages in WT nerve are elongated at 5 days post-cut. FIG. 3B includes an electron micrograph and shows that undigested myelin debris and ovoids are present in WT nerve at 5 days post-crush. FIG. 3C includes a stain for myelin basic protein (MBP) and shows WT nerve with abundant elongated MBP-positive myelin debris at 5 days post-cut. FIG. 3D includes a stain for Iba1 and shows that Iba1-positive BACE1 KO macrophages are larger in size and appear activated at 5 days post-cut. FIG. 3E includes an electron micrograph and shows that activated macrophages filled with lipid droplets (arrow) are present in BACE1 KO nerves at 5 days post-crush. FIG. 3F includes a stain for MBP and shows BACE1 KO nerve with broken down MBP-positive myelin debris at 5 days post-cut. FIGS. 3G and 3H include brightfield images of cultured peritoneal macrophages. FIG. 3G shows WT macrophages after incubation with IgG-coated beads for 5 minutes. FIG. 3H shows BACE1 KO macrophages after incubation with IgG-coated beads for 5 minutes. FIG. 3I includes a graph showing quantification of IgG-coated beads per individual cells following 5 minutes of phagocytosis. Data are from 3 independent experiments, and values are mean±SEM. Scale bars=10 μm in FIGS. 3A and 3D, 2 μm in FIGS. 3B and 3E, 20 μm in FIGS. 3C and 3F, and 100 μm in FIGS. 3G and 3H.

FIGS. 4A-4D show accelerated rate of axonal outgrowth in BACE1 KO vs. WT following crush injury. FIGS. 4A and 4B include images of longitudinally sectioned sciatic nerves stained for GAP43 at 3 days post-crush. In a 2 mm segment distal to the crush, BACE1 KO nerve (FIG. 4B) has more regenerating axons that reach further than those axons of WT littermate nerve (FIG. 4A). FIGS. 4C and 4D include images of sections of nerves filled with neurobiotin to anterogradely label regenerating axons at 5 days post-crush. BACE1 KO nerve (FIG. 4D) has significantly more regenerated axons that reach further distally compared to WT littermate nerve (FIG. 4C). Scale bars in FIGS. 4A and 4B=100 μm and in FIGS. 4C and 4D=500 μm. Stars in FIGS. 4C and 4D indicate approximate sites of crush.

FIGS. 5A-5G show extensive axonal regeneration and large polyaxonal pockets in the distal segment of crushed BACE1 KO sciatic nerve at 5 days post-crush. FIGS. 5A-5F are electron micrographs. FIG. 5A shows abundant myelin debris and ovoids and few regenerating sprouts in WT nerve (arrow). FIG. 5B shows that myelin debris is cleared and numerous regenerating sprouts (arrows) are present in BACE1 KO nerve. FIG. 5C shows few polyaxonal pockets are present in WT nerve. FIG. 5D shows numerous polyaxonal pockets (arrows) are present in BACE1 KO nerve. FIG. 5E shows a degenerating myelinated fiber in WT nerve that has one (arrow) sprout growing beneath the basal lamina membrane. FIG. 5F shows that multiple sprouts (arrows) are growing within individual degenerating fibers in BACE1 KO nerve. FIG. 5G includes a graph showing that BACE1 KO fibers exhibit enhanced number of regenerating sprouts within a single basal lamina membrane compared to WT fibers. For example, 66% of the BACE1 KO fibers have 2 or more regenerating sprouts compared to 31% of the WT fibers, which have 2 or more sprouts. Scale bars=2 μm in FIGS. 5A-5D.

FIGS. 6A and 6B show that BACE1 KO regenerating axons form a central channel of microtubule cluster whereas WT axons do not. FIGS. 6A and 6B include images of the central channel of a microtubule cluster in regenerating WT (FIG. 6A) and BACE1 KO (FIG. 6B) axons at 10 days post-crush. FIG. 6A shows regenerated axon with normally distributed cytoskeleton structures such as microtubules and neurofilament. FIG. 6B shows regenerated BACE1 KO axon with a central channel of microtubules.

FIGS. 7A-7G show that significantly more regenerated axons are present at 10 and 15 days post-crush in the distal segments of crushed BACE1 KO vs. WT sciatic nerve. FIGS. 7A-7D include images of semi-thin (1 μm) plastic sections stained with toluidine blue. FIG. 7A shows a large amount of myelin debris in various stages of degradation with some regenerating axons in WT nerve 10 days post-crush. FIG. 7B shows a large number of regenerating axons, some foamy macrophages, and myelin debris in BACE1KO nerve 10 days post-crush. FIG. 7C shows regenerating axons in WT nerve 15 days post-crush. FIG. 7D shows significantly more regenerating axons in BACE1 KO nerve 15 days post-crush (compare FIGS. 7C and 7D). FIGS. 7E and 7F are images of frozen sections stained for phosphorylated neurofilament (NF 160). FIG. 7E shows a few NF 160-positive axons that are regenerating in a WT nerve segment at 10-12 mm distal to the crush site. FIG. 7F shows significant numbers of NF 160-positive axons regenerating in a BACE1 KO nerve segment at 10-12 mm distal to the crush site. FIG. 7G includes a graph showing axon counts at 10 and 15 days post-crush. At both time points, BACE1 KO nerves have significantly more regenerated axons than WT nerves. *p<0.05. n=3 nerve per genotype at each time point. Values are mean±SEM. Scale bar=10 μm for FIGS. 7A-7D.

FIGS. 8A-8G show robust reinnervation of neuromuscular junctions in BACE1 KO vs. WT at 10 days post-crush. 10 days after nerve crush, the gastrocnemius muscle was harvested from BACE1 KO and WT mice. FIGS. 8A and 8B include images of samples stained for synaptophysin. FIGS. 8C and 8D include images of samples stained for cc bungarotoxin. FIGS. 8E and 8F are merged images of (FIGS. 8A and 8C) and (FIGS. 8B and 8D), respectively. Re-innervating pre-synaptic endings at neuromuscular junctions in BACE1 KO appeared more mature than those in WT. FIG. 8G includes a graph showing counts of neuromuscular junctions in WT and BACE1 KO mice. BACE1 KO muscles have significantly more reinnervated junctions. N=3 per genotype. Values are mean±SEM. *p<0.05.

FIGS. 9A-9G show that BACE1 inhibitors accelerate debris clearance and axonal regeneration following axotomy in WT mice. FIGS. 9A and 9B include images that are projections of Z-stacks from whole mounted-nerves to show the YFP-positive axonal debris through the depth of the nerve. FIG. 9A shows YFP-WT nerve at 7 days post-crush. Fragmented YFP debris (arrowheads) is present, and few regenerated YFP-positive axons (arrows) are growing beneath the debris. FIG. 9B shows YFP-WT nerve treated with BACE1 inhibitor IV for 7 days following nerve crush. YFP debris (arrowheads) is more fragmented in the nerve, and numerous regenerated YFP-positive axons (arrows) are growing beneath the debris. FIG. 9C includes an electron micrograph of WT nerve at 7 days post-crush. A few regenerating axons (arrows) are present. FIG. 9D includes an electron micrograph of WT nerve treated with BACE1 inhibitor IV at 7 days post-crush. The nerve has more regenerated axons (arrows) than WT littermate nerve (see FIG. 9C). More post-phagocytic macrophages (arrowhead) are also present. FIG. 9E includes an image of WT foot stained for β tubulin III to reveal the intra-plantar nerves. FIG. 9F includes an image of WT foot stained for β tubulin III 2 weeks after sciatic nerve crush. Regenerated axons have not arrived at the foot yet. FIG. 9G includes an image of WT foot stained for β tubulin III 2 weeks after sciatic nerve crush and treatment with BACE1 inhibitor WAY 258131. β tubulin III positive fibers are regenerated axons in the inta-planter nerves. Scale bars in FIGS. 9A and 9B=100 μm and in FIGS. 9C and 9D=2 μm.

FIGS. 10A-10D show accelerated axonal regeneration of BACE1 KO axons. FIG. 10A includes a schematic representation of a reciprocal sciatic nerve transplantation between BACE1 KO and WT (above). FIG. 10A also includes a representative image of a nerve that was transplanted using this nerve graft technique (below). FIG. 10B includes an image of a WT nerve segment transplanted into a BACE1 KO host nerve. BACE1 KO axons regenerated through the WT graft well. Arrows point to regenerated BACE1 KO axons. FIG. 10C includes an image of a BACE1 nerve segment transplanted into a wild-type host. A few WT axons grow through the BACE1 graft (arrow). Scale bars for FIGS. 10B and 10C=10 μm. FIG. 10D includes a graph showing that BACE1 KO DRG explants had axons 119.0% and 150.0% longer on average than those of WT DRG explants at 2 and 4 days post culture. BACE1 inhibitor IV (50 nM) increased axonal outgrowth of WT DRG explants by 98.0% at 4 days post culture. Values are mean±SEM*p<0.001.

### DETAILED DESCRIPTION OF THE INVENTION

This invention is based, at least in part, on the discovery that BACE1 is a therapeutic target for nerve injuries.

As discussed in detail below, axonal degeneration and axonal regeneration in adult nerves are affected by inhibition or elimination of BACE1. BACE1 knockout and wild-type nerves degenerate at a similar rate after axotomy and to a similar extent in the experimental neuropathies produced by administration of paclitaxel and acrylamide. Unexpectedly, however, it was discovered that BACE1 knockout mice had markedly enhanced clearance of axonal and myelin debris from degenerated fibers, accelerated axonal regeneration, and earlier reinnervation of neuromuscular junctions as compared to littermate controls. These observations indicate that BACE1 inhibition provides a novel therapeutic approach for accelerating regeneration and recovery after nerve damage.

### BACE1

β-site amyloid precursor protein cleaving enzyme 1 (BACE1) is a 501 amino acid protein that bears homology to eukaryotic aspartic proteases. Like other aspartic proteases, BACE1 is synthesized as a zymogen with a pro-domain that is cleaved by furin to release the mature protein. BACE1 is a type I transmembrane protein with a lumenal active site that cleaves APP to release N-APP into the extracellular space. The remaining carboxyl-terminal fragment undergoes subsequent cleavage by γ-secretase to generate amyloid-β peptides.

Full nucleotide sequences encoding BACE1, and variants related thereto, are well-known in the art. The BACE1 gene is conserved in humans, non-human primates, canines, bovines, murines, rodents, avians, and the like, and exemplary sequences of human BACE1 are available under NCBI Accession Nos. NM_{—}138971, NM_{—}138972, NM_{—}138973, and NM_{—}012104.

As described in detail below, BACE1 is a novel therapeutic target for treating nerve injury. Inhibition of BACE1 expression and biological activity enhances axonal regeneration and enhances reinnervation of tissue. Accordingly, the present invention provides methods that are useful for reducing the expression and/or biological activity of BACE1.

### BACE1 Inhibitors

The invention provides for the use of one or more agents to reduce the expression and/or biological activity of BACE1. The agent can be any peptide, polypeptide, polynucleotide, or small molecule that is capable of reducing the expression and/or biological activity of BACE1

In one aspect of the invention, the agent is a small molecule that reduces the expression and/or biological activity of BACE1. Small molecule BACE1 inhibitors are well-known in the art, and examples of such agents are disclosed in Citron, Trends Pharmacol. Sci. 25:92-97 (2004) and Silvestri, Med. Res. Rev. 29:295-338 (2008), which are hereby incorporated by reference. Exemplary agents include, but are not limited to, a peptidomimetic inhibitor of BACE1, a non-peptidomimetic inhibitor of BACE1, a macrocyclic inhibitor of BACE1, BACE1 inhibitor IV, OM99-2, OM00-3, GT-1017, KMI-429, KMI-570, KMI-684, CTS-2116, WAY-258131, AZ29, and a pharmaceutically acceptable salt or prodrug thereof. Additional agents include CTS-2116 (CoMentis); WAY-258131 (Wyeth); AZ29 (Genentech); and BACE1 inhibitors by Johnson & Johnson, Eisai, Merck, Schering-Plough, and TransTech Pharma.

In another aspect of the invention, the agent is a nucleic acid molecule that reduces the expression and/or biological activity of BACE1. Such oligonucleotides are well-known in the art and include single and double stranded nucleic acid molecules (e.g., DNA, RNA, and analogs thereof) that bind a nucleic acid molecule that encodes BACE1 (e.g., antisense molecules, siRNA, shRNA) as well as nucleic acid molecules that bind directly to BACE1 to modulate its biological activity (e.g., aptamers). See, e.g., Narwot, Acta Biochimica Polonica 51:431-444 (2004), which is hereby incorporated by reference.

In another aspect of the invention, the agent is an antibody or antibody fragment that specifically binds to BACE1 and reduces the expression and/or biological activity of BACE1, or specifically binds to APP and prevents BACE1 cleavage of APP. Such antibodies and antibody fragments are well-known in the art. As described in detail below, methods for making and screening such antibodies and antibody fragments are also within the purview of the skilled artisan.

Ribozymes

Catalytic RNA molecules or ribozymes that include an antisense BACE1 sequence of the present invention can be used to inhibit expression of a BACE1 nucleic acid molecule in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988) and U.S. Patent Application Publication No. 2003/0003469 A1, each of which is hereby incorporated by reference.

Accordingly, the invention also features a catalytic RNA molecule that includes, in the binding arm, an antisense RNA having between eight and nineteen consecutive nucleobases. In embodiments, the catalytic nucleic acid molecule is formed in a hammerhead or hairpin motif. Examples of such hammerhead motifs are described by Rossi et al., Aids Research and Human Retroviruses 8:183 (1992). Examples of hairpin motifs are described in U.S. Pat. No. 5,866,701; Hampel and Tritz, Biochemistry 28:4929 (1989); and Hampel et al., Nucleic Acids Research 18:299 (1990). These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs.

While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

siRNA

Short twenty-one to twenty-five nucleotide double-stranded RNAs are effective at down-regulating gene expression. (Zamore et al., Cell 101:25-33 (2000); and Elbashir et al., Nature 411:494-498 (2001)). The therapeutic effectiveness of an sirNA approach in mammals was demonstrated in vivo by McCaffrey et al. Nature 418:38-39 (2002).

Given the sequence of a target gene, siRNAs may be designed to inactivate that gene. Such siRNAs, for example, could be administered directly to an affected tissue, or administered systemically. The nucleic acid sequence of a BACE1 gene can be used to design small interfering RNAs (siRNAs). The 21 to 25 nucleotide siRNAs may be used, for example, as therapeutics to treat a nerve injury.

The inhibitory nucleic acid molecules of the present invention may be employed as double-stranded RNAs for RNA interference (RNAi)-mediated knock-down of BACE1 expression. In embodiments, BACE1 expression is reduced in a neuron. RNAi is a method for decreasing the cellular expression of specific proteins of interest (reviewed in Tuschl, Chembiochem. 2:239-245 (2001); Sharp, Genes & Devel. 15:485-490 (2000); Hutvagner and Zamore, Curr. Opin. Genet. Devel. 12:225-232 (2002); and Hannon, Nature 418:244-251 (2002). The introduction of siRNAs into cells either by transfection of dsRNAs or through expression of siRNAs using a plasmid-based expression system is increasingly being used to create loss-of-function phenotypes in mammalian cells.

In embodiments, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al., Science 296:550-553 (2002); Paddison et al., Genes & Devel. 16:948-958 (2002); Paul et al., Nature Biotechnol. 20:505-508 (2002); Sui et al., Proc. Natl. Acad. Sci. USA 99:5515-5520 (2002); Yu et al., Proc. Natl. Acad. Sci. USA 99:6047-6052 (2002); Miyagishi et al., Nature Biotechnol. 20:497-500 (2002); and Lee et al., Nature Biotechnol. 20:500-505 (2002), which are hereby incorporated by reference.

Small hairpin RNAs (shRNAs) comprise an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well-known in the art and the term is used consistently with its known meaning in the art. As is known in the art, the secondary structure does not require exact base-pairing. Thus, the stem can include one or more base mismatches or bulges. Alternatively, the base-pairing can be exact, i.e., not include any mismatches. The multiple stem-loop structures can be linked to one another through a linker, such as, for example, a nucleic acid linker, a miRNA flanking sequence, other molecule, or some combination thereof.

As used herein, the term “small hairpin RNA” includes a conventional stem-loop shRNA, which forms a precursor miRNA (pre-miRNA). While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some instances the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

In this regard, short hairpin RNAs can be designed to mimic endogenous miRNAs. Many miRNA intermediates can be used as models for shRNA or shRNAmir, including without limitation an miRNA comprising a backbone design of miR-15a, -16, -19b, -20, -23a, -27b, -29a, -30b, -30c, -104, -132s, -181, -191, -223 (see U.S. Publication No. 2005/0075492). In embodiments, shRNA molecules are designed based on the human miR-30 sequence, redesigned to allow expression of artificial shRNAs by substituting the stem sequences of the pri-miR-30 with unrelated base-paired sequences (see Siolas et al., Nat. Biotech. 23:227-231 (2005); Silva et al., Nat. Genet. 37:1281-1288 (2005); Zeng et al., Molec. Cell 9:1327-1333 (2002)). The natural stem sequence of the miR-30 can be replaced with a stem sequence from about 16 to about 29 nucleotides in length, in particular from about 19 to 29 nucleotides in length. The loop sequence can be altered such that the length is from about 4 to about 23 nucleotides. In embodiments, the stem of the shRNA molecule is about 22 nucleotides in length. In other embodiments, the stem is about 29 nucleotides in length. Thus, the invention can be practiced using shRNAs that are synthetically produced, as well as microRNA (miRNA) molecules that are found in nature and can be remodeled to function as synthetic silencing short hairpin RNAs.

shRNAs can be expressed from DNA vectors to provide sustained silencing and high yield delivery into almost any cell type. In embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. A retroviral plasmid vector can be employed to transduce packaging cell lines to form producer cell lines. Examples of packaging cells that can be transfected include, but are not limited to, the PE501, PA317, R-2, R-AM, PA12, T19-14x, VT-19-17-H2, RCRE, RCRIP, GP+E-86, GP+envAm12, and DAN cell lines as described in Miller, Human Gene Therapy 1:5-14 (1990), which is incorporated herein by reference. The vector can transduce the packaging cells through any means known in the art. A producer cell line generates infectious retroviral vector particles which include polynucleotide encoding a DNA replication protein. Such retroviral vector particles can then be employed to transduce eukaryotic cells either in vitro or in vivo. The transduced eukaryotic cells will express a DNA replication protein.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., Nat. Genet. 39: 914-921 (2005)). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Publication No. WO 2004/029219 A2 and Fewell et al., Drug Discovery Today 11:975-982 (2006) for a description of inducible shRNA.

### Antibodies and Antibody Fragments

The antibody, or antibody fragment, can be any monoclonal or polyclonal antibody or antibody fragment that specifically binds to BACE1 and reduces the expression or biological activity of BACE1. The antibody, or antibody fragment, can be any monoclonal or polyclonal antibody or antibody fragment that specifically binds to a protein that interacts with BACE1. For example, the antibody or antibody fragment can bind to APP and block BACE1 cleavage. See Nikolaev et al., Nature 457: 981-9 (2009); Arbel et al., Proc Natl Acad Sci USA 102: 7718-23 (2005); and Arbel & Solomon, Curr Alzheimer Res 4: 437-45 (2007), which are hereby incorporated by reference.

Methods for preparing polyclonal antibodies are well-known in the art. Polyclonal antibodies can be raised by immunizing an animal (e.g., a rabbit, rat, mouse, donkey, and the like) with multiple subcutaneous or intraperitoneal injections of the relevant antigen (a purified peptide fragment, full-length recombinant protein, fusion protein, and the like) optionally conjugated to keyhole limpet hemocyanin (KLH), serum albumin, and the like, diluted in sterile saline and combined with an adjuvant (e.g. Complete or Incomplete Freund's Adjuvant) to form a stable emulsion. The polyclonal antibody is then recovered from blood, ascites, and the like, of an animal so immunized. Collected blood is clotted, and the serum decanted, clarified by centrifugation, and assayed for antibody titer. The polyclonal antibodies can be purified from serum or ascites according to standard methods in the art, including, but not limited to, affinity chromatography, ion-exchange chromatography, gel electrophoresis, dialysis.

Monoclonal antibodies can be prepared using hybridoma methods, such as those described by Kohler and Milstein, Nature 256:495 (1975). Using the hybridoma method, a mouse, hamster, or other appropriate host animal, is immunized as described above to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated either in vitro culture using standard methods (see Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively monoclonal antibodies can also be made using recombinant DNA methods well-known in the art. See, e.g., U.S. Pat. No. 4,816,567. For example, polynucleotides encoding a monoclonal antibody can be isolated from mature B-cells, hybridoma cells, and the like, using RT-PCR oligonucleotide primers that specifically amplify the genes encoding the heavy and light chains of the antibody. The sequence is determined using conventional procedures, and the isolated polynucleotides encoding the heavy and light chains are then cloned into suitable expression vectors. Transfection of the expression vectors into host cells such as E. coli cells, simian COS cells, Chinese hamster ovary (CHO) cells, myeloma cells, and the like, results in generation of the monoclonal antibody of interest by the host cells. Also, recombinant monoclonal antibodies or fragments thereof of the desired species can be isolated from phage display libraries as described in McCafferty et al., Nature 348:552-554 (1990); Clackson et al., Nature 352:624-628 (1991); and Marks et al., J. Mol. Biol. 222:581-597 (1991).

The polynucleotide(s) encoding a monoclonal antibody can further be modified in a number of different manners using recombinant DNA technology to generate alternative antibodies. In embodiments, the constant domains of the light and heavy chains of, for example, a mouse monoclonal antibody can be substituted 1) for those regions of, for example, a human antibody to generate a chimeric antibody, or 2) for a non-immunoglobulin polypeptide to generate a fusion antibody. In embodiments, the constant regions are truncated or removed to generate the desired antibody fragment of a monoclonal antibody. Furthermore, site-directed or high-density mutagenesis of the variable region can be used to optimize specificity, affinity, etc. of a monoclonal antibody.

In embodiments, the monoclonal antibody against BACE1 is a humanized antibody. Humanized antibodies are antibodies that contain minimal sequences from non-human (e.g murine) antibodies within the variable regions. Such antibodies are used therapeutically to reduce antigenicity and HAMA (human anti-mouse antibody) responses when administered to a human subject. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the CDR of a human antibody with that of a non-human antibody (e.g., mouse, rat, rabbit, hamster, and the like) having the desired specificity, affinity, and capability (see Jones et al., Nature 321:522-525 (1986); Riechmann et al., Nature 332:323-327 (1988); and Verhoeyen et al., Science 239:1534-1536 (1988)). The humanized antibody can be further modified by the substitution of additional residues either in the Fv framework region and/or within the replaced non-human residues to refine and optimize antibody specificity, affinity, and/or capability.

Human antibodies can be directly prepared using various techniques well-known in the art. Immortalized human B lymphocytes immunized in vitro or isolated from an immunized individual that produces an antibody directed against a target antigen can be generated (See, e.g., Cole et al., Monoclonal Antibodies and Cancer Therapy, Alan R. Liss, p. 77 (1985); Boemer et al., J. Immunol. 147:86-95 (1991); and U.S. Pat. No. 5,750,373). Also, the human antibody can be selected from a phage library that expresses human antibodies (see Vaughan et al., Nature Biotechnology 14:309-314 (1996); Sheets et al., PNAS 95:6157-6162 (1998); Hoogenboom and Winter, J. Mol. Biol. 227:381 (1991); and Marks et al., J. Mol. Biol. 222:581 (1991)). Human antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

It may further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

The invention further embraces variants and equivalents which are substantially homologous to the chimeric, humanized and human antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid. What is intended by a conservative amino acid substitution is well known in the art.

### Methods of the Invention

Methods of the invention address a long felt need for therapeutics useful for enhancing axonal regeneration following injury of the CNS or PNS, or any condition or disorder characterized by degeneration of axons. These methods can be used to enhance axonal outgrowth, enhance axonal regeneration; enhance reinnervation of tissue; as well as prevent, treat, or reduce symptoms of nerve injury in a subject.

Thus, in one aspect, the invention provides methods for enhancing axonal outgrowth. In embodiments, the methods involve contacting an axon with an effective amount of an agent that reduces the expression or biological activity of BACE1.

In another aspect, the invention provides methods for enhancing axonal regeneration. In embodiments, the methods involve contacting an axon with an effective amount of an agent that reduces the expression or biological activity of BACE1.

In another aspect, the invention provides methods for enhancing reinnervation of a target tissue. In embodiments, the methods involve contacting a neuron and/or the target tissue with an effective amount of an agent that reduces the expression or biological activity of BACE1.

In another aspect, the invention provides methods for preventing, treating, or reducing symptoms of nerve injury in a subject. In embodiments, the methods involve administering to the subject an effective amount of an agent that reduces the expression or biological activity of BACE1.

In another aspect, the invention provides methods for increasing nerve regeneration in a mammalian subject after peripheral nerve injury. In embodiments, the methods involve administering to the subject an effective amount of an agent that reduces the expression or biological activity of BACE1. In embodiments, the methods further involve comparing the amount of nerve regeneration in a subject after administration of the agent and modifying the amount and time of administration so that nerve regeneration is increased in the subject as compared to a control.

In embodiments, the agent is a peptide, polypeptide, polynucleotide, or small molecule. In related embodiments, the agent is an inhibitory nucleic acid molecule (e.g., an antisense nucleic acid molecule, an siRNA, or an shRNA that reduces the expression of BACE1). In related embodiments, the agent is an antibody that specifically binds to BACE1. In related embodiments, the agent is a small molecule (e.g., a peptidomimetic inhibitor of BACE1, a non-peptidomimetic inhibitor of BACE1, a macrocyclic inhibitor of BACE1, BACE1 inhibitor IV, OM99-2, OM00-3, GT-1017, KMI-429, KMI-570, KMI-684, CTS-2116, WAY-258131, AZ29, and a pharmaceutically acceptable salt or prodrug thereof). In related embodiments, the agent is a BACE1 inhibitor IV. The agent can also be any peptide, polypeptide, polynucleotide, or small molecule described herein that is capable of reducing the expression or biological activity of BACE1.

In embodiments, the agent is a combination of any of the agents and/or compounds described herein.

In embodiments, about 50 nM to about 1 μM of the agent is administered to a subject.

In embodiments, the subject is a rodent, bovine, feline, or canine. In embodiments, the subject is human. In related embodiments, the subject has been identified as suffering from nerve injury.

The methods described herein are particularly useful for treating a subject suffering from nerve injury resulting from physical injury, ischemia, neurological disorders, certain medical procedures or therapies, tumors, infections, metabolic or nutritional disorders, cognition or mood disorders, and the like. Exemplary injuries include, but are not limited to, physical injuries, traumatic brain injury, spinal cord injury, ischemic injury, and neuropathies (e.g., diabetic neuropathy and chemotherapy-induced neuropathy). Such injuries are associated with axonal degeneration.

In embodiments, the methods described herein are also useful for treating a subject suffering from disorders associated with denervation and atrophy of muscle. For example, axonal degeneration and axonal regeneration have been addressed in disorders of demyelination with secondary axonal degeneration (e.g., Charcot-Marie-Tooth disease type 1 (CMT1). Enhancing axonal regeneration, e.g., by administering a BACE1 inhibitor, will lessen the symptoms of the these disorders.

The therapeutic efficacy of the methods of the invention can optionally be assayed by measuring (i) the formation of new presynaptic terminals; (ii) an increase in myelin debris removal; and/or (iii) re-innervation of neuromuscular junctions. Methods for these parameters are standard in the art and are described herein.

### Pharmaceutical Compositions

The invention provides for pharmaceutical compositions containing at least one agent that reduces the expression and/or biological activity of BACE1. The agent can be any one of such agents described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for enhancing axonal outgrowth, enhancing axonal regeneration; enhancing reinnervation of tissue; as well as preventing, treating, or reducing symptoms of nerve injury in a subject.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well-known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

### Methods of Delivery

The pharmaceutical compositions of the invention can be administered to a subject by oral and non-oral means (e.g., topically, transdermally, or by injection). Such modes of administration and the methods for preparing an appropriate pharmaceutical composition for use therein are described in Gibaldi's Drug Delivery Systems in Pharmaceutical Care (1st ed., American Society of Health-System Pharmacists), which is hereby incorporated by reference.

In embodiments, the pharmaceutical compositions are administered orally in a solid form.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well-known in the art.

The pharmaceutical compositions can also be formulated so as to provide slow, extended, or controlled release of the active ingredient therein using, for example, hydroxypropylmethyl cellulose in varying proportions to provide the desired release profile, other polymer matrices, liposomes and/or microspheres. The pharmaceutical compositions can also optionally contain opacifying agents and may be of a composition that releases the active ingredient(s) only, or preferentially, in a certain portion of the gastrointestinal tract, optionally, in a delayed manner. Examples of embedding compositions include polymeric substances and waxes. The active ingredient can also be in micro-encapsulated form, if appropriate, with one or more pharmaceutically acceptable carriers, excipients, or diluents well-known in the art (see, e.g., Remington and Remington's).

The pharmaceutical compositions can be sterilized by, for example, filtration through a bacteria-retaining filter, or by incorporating sterilizing agents in the form of sterile solid compositions which can be dissolved in sterile water, or some other sterile injectable medium immediately before use.

In embodiments, the pharmaceutical compositions are administered orally in a liquid form.

Liquid dosage forms for oral administration of an active ingredient include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. In addition to inert diluents, the liquid pharmaceutical compositions can include adjuvants such as wetting agents, emulsifying and suspending agents, sweetening, flavoring, coloring, perfuming and preservative agents, and the like.

Suspensions, in addition to the active ingredient(s) can contain suspending agents such as, but not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof.

In embodiments, the pharmaceutical compositions are administered by non-oral means such as by topical application, transdermal application, injection, and the like. In related embodiments, the pharmaceutical compositions are administered parenterally by injection, infusion, or implantation (e.g., intravenous, intramuscular, intraarticular, subcutaneous, and the like).

Compositions for parenteral use can be presented in unit dosage forms, e.g. in ampoules or in vials containing several doses, and in which a suitable preservative can be added. Such compositions can be in form of a solution, a suspension, an emulsion, an infusion device, a delivery device for implantation, or it can be presented as a dry powder to be reconstituted with water or another suitable vehicle before use. One or more co-vehicles, such as ethanol, can also be employed. Apart from the active ingredient(s), the compositions can contain suitable parenterally acceptable carriers and/or excipients or the active ingredient(s) can be incorporated into microspheres, microcapsules, nanoparticles, liposomes, or the like for controlled release. Furthermore, the compositions can also contain suspending, solubilising, stabilising, pH-adjusting agents, and/or dispersing agents.

The pharmaceutical compositions can be in the form of sterile injections. To prepare such a composition, the active ingredient is dissolved or suspended in a parenterally acceptable liquid vehicle. Exemplary vehicles and solvents include, but are not limited to, water, water adjusted to a suitable pH by addition of an appropriate amount of hydrochloric acid, sodium hydroxide or a suitable buffer, 1,3-butanediol, Ringer's solution and isotonic sodium chloride solution. The pharmaceutical composition can also contain one or more preservatives, for example, methyl, ethyl or n-propyl p-hydroxybenzoate. To improve solubility, a dissolution enhancing or solubilising agent can be added or the solvent can contain 10-60% w/w of propylene glycol or the like.

The pharmaceutical compositions can contain one or more pharmaceutically acceptable sterile isotonic aqueous or nonaqueous solutions, dispersions, suspensions or emulsions, or sterile powders, which can be reconstituted into sterile injectable solutions or dispersions just prior to use. Such pharmaceutical compositions can contain antioxidants; buffers; bacteriostats; solutes, which render the formulation isotonic with the blood of the intended recipient; suspending agents; thickening agents; preservatives; and the like.

Examples of suitable aqueous and nonaqueous carriers, which can be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well-known in the art, including transscrotal patches. Pharmaceutical compositions in such transdermal patches can contain one or more absorption enhancers or skin permeation enhancers well-known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, Transcutol®, and Azone®.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well-known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as Silastic™, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilising agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the ratecontrolling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient ance onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the above-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

### Dosages

When the agents described herein are administered as pharmaceuticals to humans and animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an effective amount or quantity sufficient to enhance axonal outgrowth, enhance axonal regeneration; enhance reinnervation of tissue; and/or prevent, treat, or reduce symptoms of nerve injury in a subject.

Exemplary dose ranges include 0.01 mg to 250 mg per day, 0.01 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In embodiments, the agent is administered at a concentration of about 10 micrograms to about 100 mg per kilogram of body weight per day, about 0.1 to about 10 mg/kg per day, or about 1.0 mg to about 10 mg/kg of body weight per day.

In embodiments, the pharmaceutical composition comprises an agent in an amount ranging between 1 and 10 mg, such as 1, 2, 3, 4, 5, 6, 7, 8, 9, or 10 mg.

In embodiments, the therapeutically effective dosage produces a serum concentration of an agent of from about 0.1 ng/ml to about 50-100 μg/ml. The pharmaceutical compositions typically should provide a dosage of from about 0.001 mg to about 2000 mg of compound per kilogram of body weight per day. For example, dosages for systemic administration to a human patient can range from 1-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

In embodiments, about 50 nM to about 1 μM of an agent is administered to a subject. In related embodiments, about 50-100 nM, 50-250 nM, 100-500 nM, 250-500 nM, 250-750 nM, 500-750 nM, 500 nM to 1 μM, or 750 nM to 1 μM of an agent is administered to a subject.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., enhanced axonal regeneration) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

### Combination Therapies

The agents and pharmaceutical compositions described herein can also be administered in combination with another therapeutic molecule. The therapeutic molecule can be any compound used to treat a subject suffering from nerve injury. Examples of such compounds include, but are not limited to, growth factors such as nerve growth factor, brain-derived neurotrophic factor, ciliary neurotrophic factor, neurotrophin-3, or glial cell-line derived neurotrophic factor; steroids such as betamethasone, dexamethasone, methylprednisolone, prednisolone, prednisone, triamcinolone, budesonide, hydrocortisone, and pharmaceutically acceptable hydrocortisone derivatives; non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam, celocoxib, refocoxib, and N-[2-(cyclohexyloxy)-4-nitrophenyl]methanesulfonamide; analgesic agents such as salicylates; sedatives such as benzodiazapines and barbiturates; antimicrobial agents such as penicillins, cephalosporins, and macrolides, including tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, minocycline, doxycycline, vancomycin, kanamycin, cephalosporins such as cephalothin, cephapirin, cefazolin, cephalexin, cephardine, cefadroxil, cefamandole, cefoxitin, cefaclor, cefuroxime, cefonicid, ceforanide, cefitaxime, moxalactam, cetizoxime, ceftriaxone, cefoperazone; and other pharmacological agents that have been shown to promote axonal regeneration, e.g., paclitaxel (Taxol®). Additional compounds include cyclosporine, a phosphodiesterase type 4 inhibitor, and dibutyryl cyclic adenosine monophosphate.

The amount of therapeutic agent administered to a subject can readily be determined by the attending physician or veterinarian. For example, it is known that Taxol® at high doses will cause axonal degeneration, but at lower doses, in combination with a BACE1 inhibitor, will counteract degeneration with enhanced regeneration.

### Kits

The invention provides for kits for enhancing axonal outgrowth, enhancing axonal regeneration; enhancing reinnervation of tissue; as well as preventing, treating, or reducing symptoms of nerve injury in a subject. In embodiments, the kit contains one or more agents or pharmaceutical compositions described herein. In embodiments, the kit provides instructions for use. The instructions for use can pertain to any of the methods described herein. In related embodiments, the instructions pertain to using the agent(s) or pharmaceutical composition(s) for enhancing axonal regeneration; enhancing reinnervation of tissue; or preventing, treating, or reducing symptoms of nerve injury in a subject. Kits according to this aspect of the invention may comprise a carrier means, such as a box, carton, tube or the like, having in close confinement therein one or more container means, such as vials, tubes, ampules, bottles and the like. In embodiments, the kit provides a notice in the form prescribed by a governmental agency regulating the manufacture, use, or sale of pharmaceuticals or biological products, which notice reflects approval by the agency of manufacture, use or sale of the kit and the components therein for human administration.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Lack of Axonal Protection in Wallerian Degeneration and in Paclitaxel- and Acrylamide-Induced Neuropathies in BACE1 KO Mice

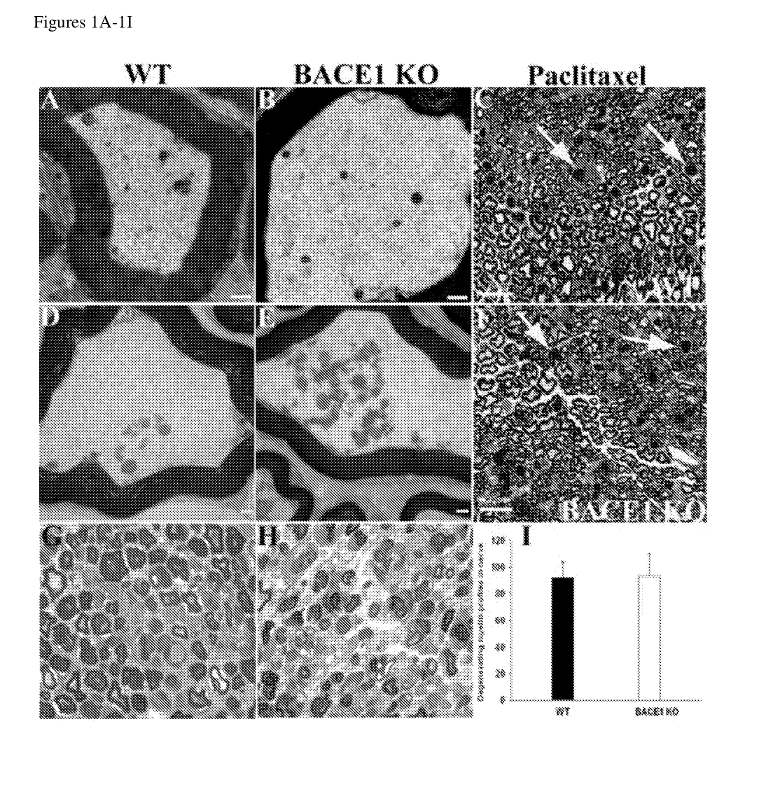

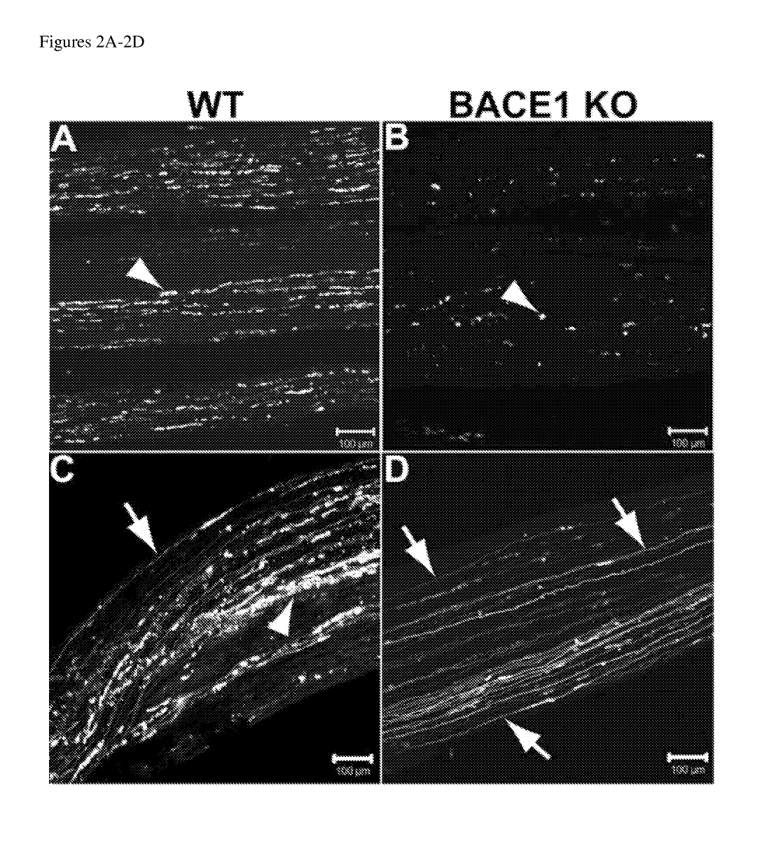

To determine whether genetic deletion of BACE1 could protect axons in adult mice in a fashion similar to that described after growth factor withdrawal during development (Nikolaev et al., Nature 457:981-9 (2009)), BACE1 knockout (KO) mice were evaluated for a delay in Wallerian degeneration after axotomy. As has been previously described (Waller, C.R. Acad. Sci. (Paris) 34:675-679 (1852), Ramon y Cajal, Degeneration and regeneration of the nervous system (Mays ed., 1913); Stoll et al., J. Neurocytol. 18:671-83 (1989); George and Griffin, J. Neurosci. 15:6445-52 (1994); Beirowski et al., J. Neurosci. Methods 134:23-35 (2004); and Beirowski et al., BMC Neurosci. 6:6 (2005)), axons and the myelin surrounding the nerve fibers in the distal segment (the nerve segment beyond the injury) were observed to undergo Wallerian degeneration after transection (FIG. 1). At 24 hours post-injury, most axons were intact, but by 48 hours post-injury, the axonal cytoskeleton and the axoplasm disintegrated into granular and amorphous debris (FIG. 1) (George and Griffin, 1994; and Bierowski et. al., 2005). In addition, the myelin sheaths in the distal stumps began to segment into characteristic ovoids. Notably, very few axons from the BACE1 KO mice or their wild type (WT) littermates exhibited an intermediate stage of degradation. Over the next several days, the axonal debris was removed from the distal stump. The initial disintegration of the axoplasm and myelin segmentation followed the same time course in BACE1 KO and WT littermate injured nerves (FIG. 1).

To examine axonal degeneration in a neuropathy model, groups of BACE1 KO and WT littermate mice were given paclitaxel, a chemotherapeutic agent that causes axonal degeneration (Roytta and Raine, J. Neurocytol. 14:157-75 (1985); Roytta and Raine, J. Neurocytol. 15:483-96 (1986)), or the neurotoxin, acrylamide (Nguyen et al., J. Neurosci. 29:630-637 (2009)). Qualitative and quantitative measures showed a similar degree of myelinated fiber degeneration in WT and BACE1 KO nerves (FIG. 1). Furthermore, unmyelinated axons degenerated comparably in both genotypes. Additionally, absence of BACE1 activity did not protect from the early axoterminal degeneration at neuromuscular junctions in BACE1 KO mice exposed to acrylamide (data not shown).

### Example 2

### Accelerated Clearance of Axonal and Myelin Debris in BACE1 KO Mice

Although the time to initiation of axonal breakdown and to myelin ovoid formation was not different, the subsequent clearance of axonal and myelin debris was markedly faster in the BACE1 KO mice. To evaluate clearance of axonal debris in BACE1 KO nerves, BACE1 KO mice were crossed with mice expressing yellow fluorescent protein (YFP) in a small subset of the axonal population (Feng et al., Neuron 28:41-51 (2000)). Line H was selected from the panel of fluorescent mice lines because it has been previously shown that a small fraction (3%) of the axons are labeled with YFP (Feng et al., 2000; Beirowski et al., Neurosci. 6:6 (2004); 2005; Vargas et al., Proc. Natl. Acad. Sci. USA 107:11993-8 (2010)). Investigating axonal degeneration in nerves marked with YFP had the advantage of allowing whole-mounts of nerves to be analyzed so that the proximal and the whole distal segment of each nerve were visualized throughout the depth of the nerves. At 7 days post-cut, little YFP-positive axonal debris remained in the BACE1 KO-YFP nerves. In contrast, much more was present in the littermate WT-YFP nerves (FIG. 2). In addition, at 10 days post crush, axonal debris was more completely cleared in BACE1 KO-YFP nerves after a crush injury than in the WT littermates (FIG. 2).

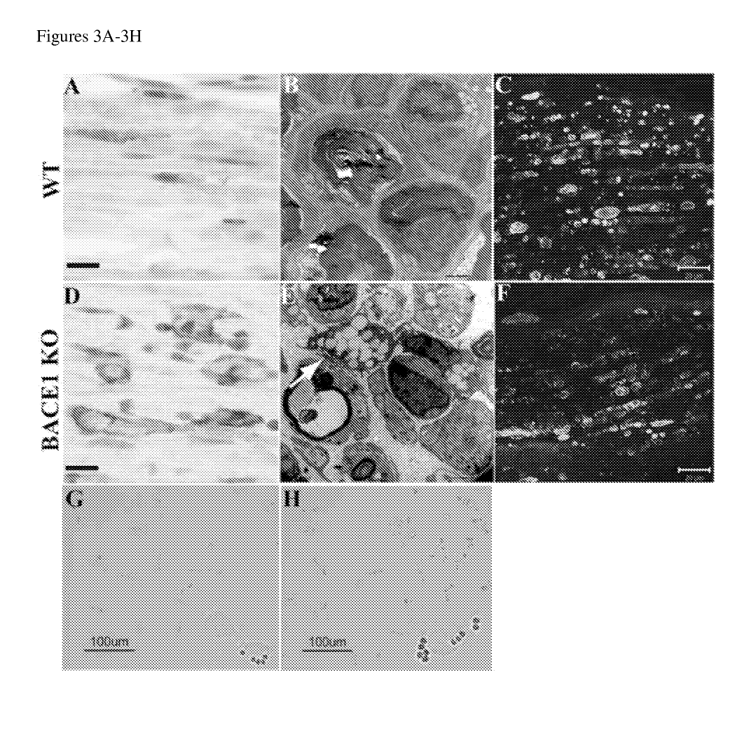

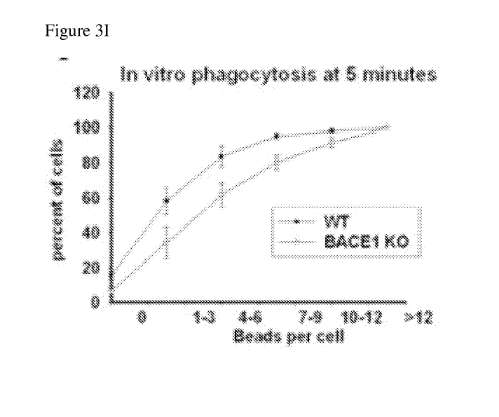

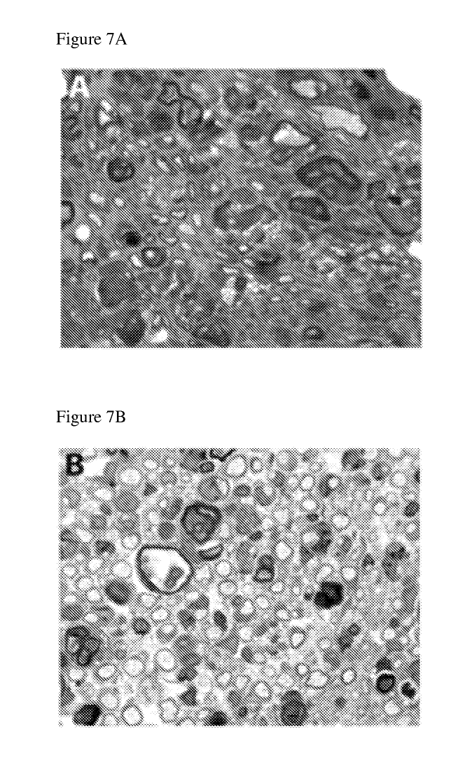

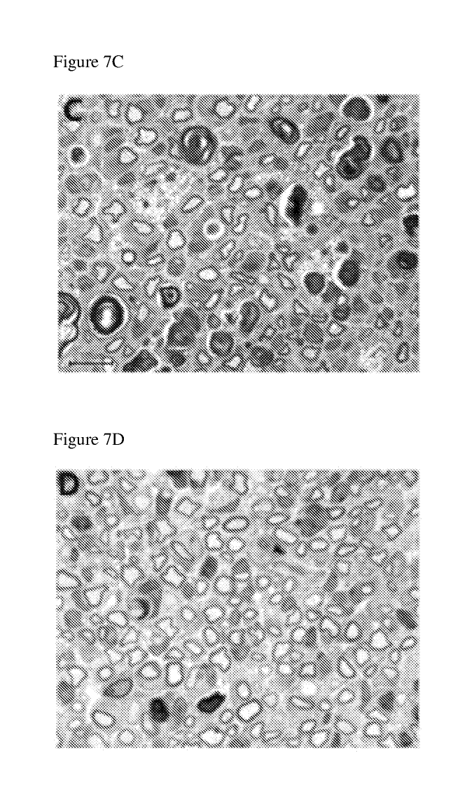

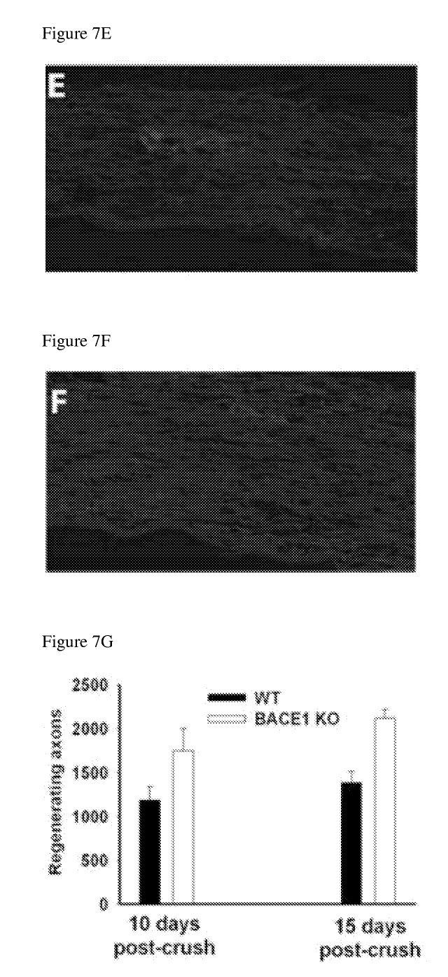

Myelin debris is known to impair axonal regeneration in the CNS, and PNS myelin has a similar effect in vitro and in vivo (Boivin et al., J. Neurosci. 28:9363-76 (2007); Barrette et al., J. Neurosci. 28:9363-76 (2008); and Vargas et al., 2010). Thus, it was determined whether reduced BACE1 activity affected the clearance of myelin debris from the distal stump. At 5 days after crush, more myelin basic protein (MBP) was degraded in BACE1 KO compared to WT as assessed by histology (FIG. 3) and Western blot (data not shown). By 15 days post-crush, clearance of myelin debris was more complete (FIG. 3, compare also FIGS. 7A-7D). One μm plastic sections showed less myelin debris in the distal stump of BACE1 KO nerves, and the remaining myelin ovoids were shorter. These differences correlated with a more rapid transition of macrophages in BACE1 KO nerves to a post-phagocytic foamy character, in which the macrophages contained numerous clear lipid droplets, representing cholesterol ester from digested myelin (FIG. 3D). By 15 days post-crush, few macrophages remained in the distal stumps of BACE1 KO mice, and those remaining were predominantly around the endoneurial blood vessels and beneath the perineurium. In contrast, distal stumps of nerves from wild-type mice still had numerous phagocytic macrophages scattered throughout the endoneurial space.

The faster clearance of axonal debris and the evolution of macrophage changes suggested a difference in phagocytosis by macrophages in the BACE1 KO animals. Electron microscopic analysis confirmed the characteristics of faster myelin degeneration in the earlier appearance of foamy post-phagocytic macrophages and the earlier clearance of myelin debris in the BACE1 KO animals (FIG. 3). To further examine macrophage behavior, noninduced peritoneal macrophages were removed and cultured. In vitro phagocytosis assays were performed as described by Link et al., Nat. Immunol. 11:232-9 (2010). This assay measures the uptake over time of beads coated with IgG as a phagocytosis index. On average, 65.5% of BACE1 KO macrophages ingested ≧4 beads per cell, and 41.8% of WT macrophages ingested ≧4 beads per cell, a difference that is statistically significant (FIGS. 3G-3I). These data indicate that BACE1 KO macrophages have enhanced phagocytic activity, which contributes to the faster in vivo clearance of myelin debris in injured BACE1 KO nerves. Although time to initial axonal degeneration was not affected by BACE1 activity, clearance of both axonal and myelin debris is accelerated in the distal stumps of BACE1 KO mice.

### Example 3

### Accelerated Axonal Regeneration in BACE1 KO Mice

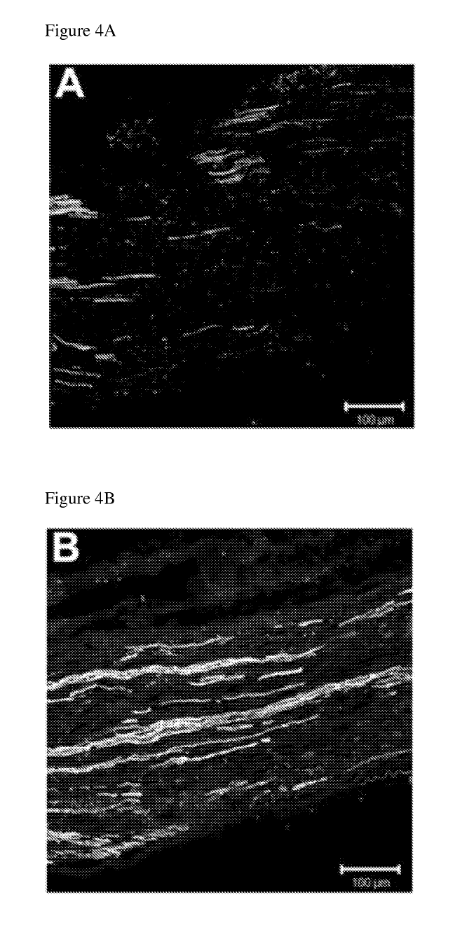

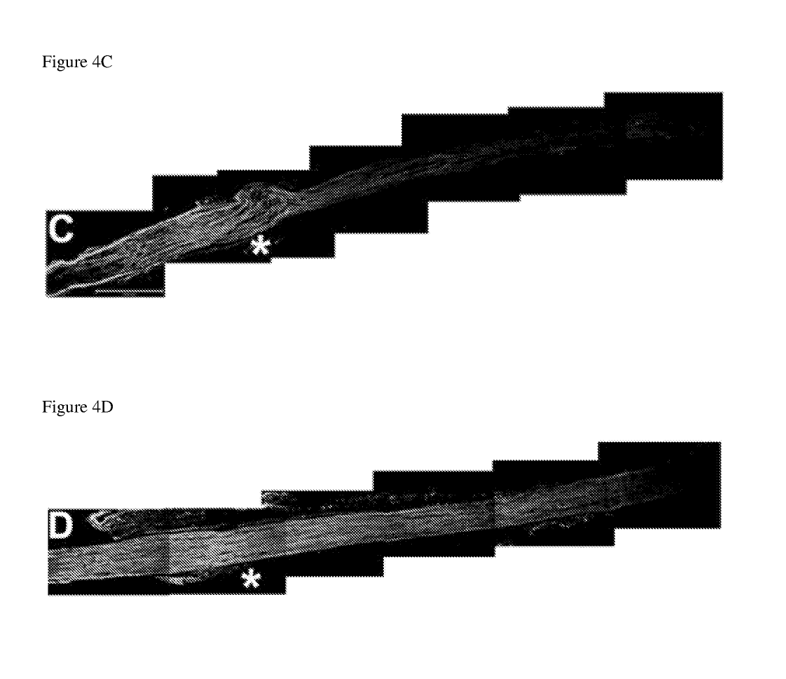

Little axonal growth occurred in either WT littermate or BACE1 KO nerves in the first 2 days after injury, as shown by GAP43 transport, neurobiotin transport, and plastic-embedded transverse sections (data not shown). As this interval represents the latency period (Lanners and Grafstein, Brain Res. 196:547-53 (1980); McQuarrie, J. Comp. Neurol. 231:239-49 (1985); Jacob et al., J. Neurobiol. 24:356-67 (1993); and Seijffers et al., J. Neurosci. 27: 7911-7920 (2007)); the data indicated that inhibition of BACE1 activity does not alter the latency period. Furthermore, the axons had shortening of the latency period in a fashion comparable to that of WT littermate axons when BACE1 KO nerves were condition lesioned (data not shown). However, by day 3 after a single nerve crush, GAP43-positive sprouts had grown farther down the distal stump in the BACE1 KO nerves than in the WT littermate nerves (FIGS. 4A and 4B). The distribution of the axonally transported marker neurobiotin was also assessed in longitudinal sections of the crushed nerves. The advantage of this exogenous labeling technique does not depend on the level of expression of endogenous genes such as GAP43. By day 5, the leading front of regenerating sprouts had grown beyond the length of sciatic nerve available in the BACE1 KO, the distal segment of the BACE1 KO nerves had significantly more regenerated axons, and these axons reached further distally, compared to WT littermate nerves (FIGS. 4C and 4D).

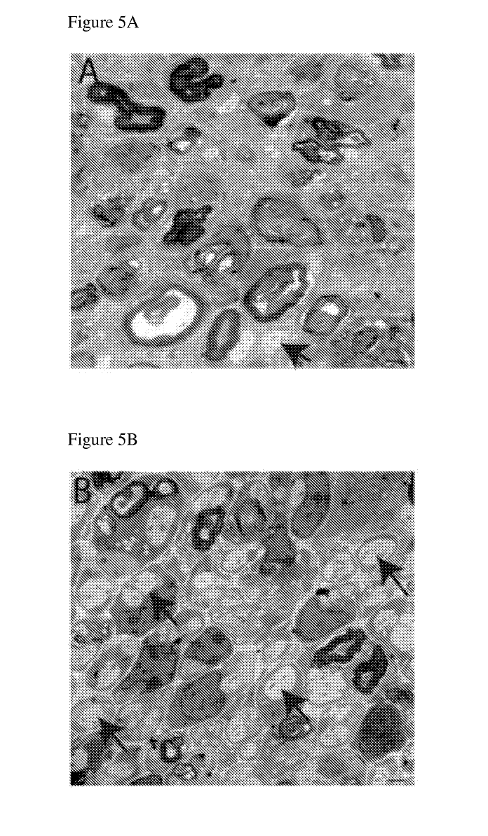

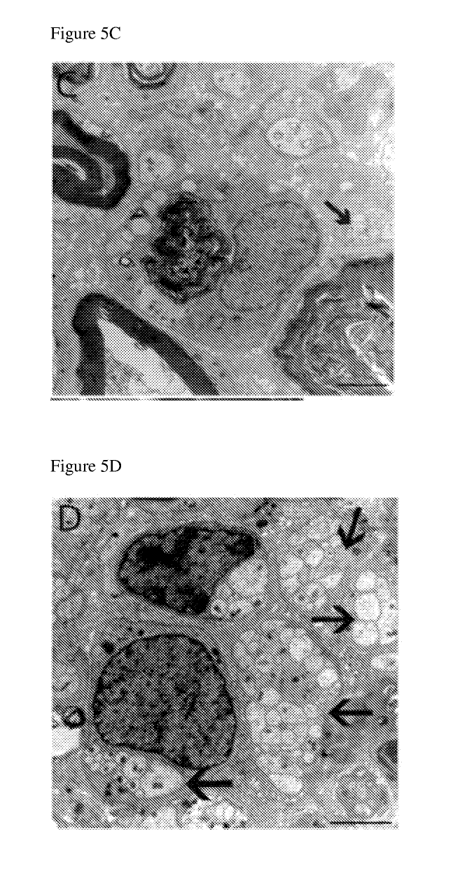

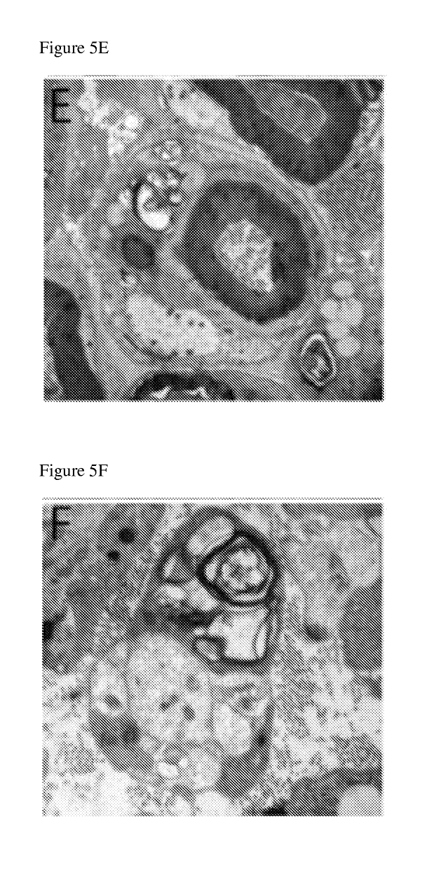

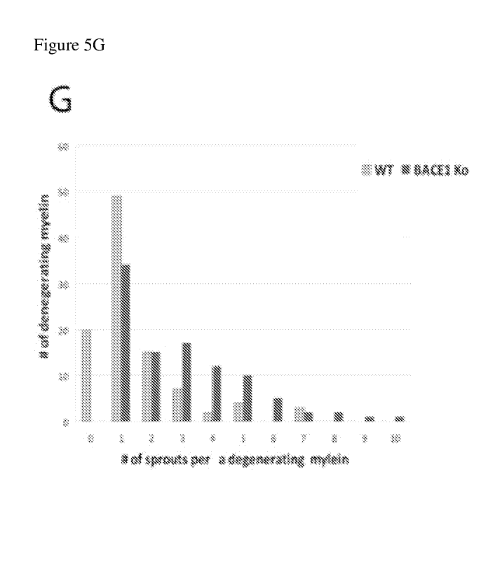

Analyses of electron micrographs confirmed that BACE KO injured axons regenerated faster than WT. In addition, more axonal sprouts were observed in the distal stump. Transverse sections of nerves were then evaluated at defined intervals 5 days after crush injury in both WT and BACE1 KO mice (FIG. 5). Significantly more axonal sprouts were observed with large (>1 μm) diameter in BACE1 KO nerve at every examined level (FIG. 5B). The sprouts with diameter <1 μm in BACE1 KO nerves were more often clustered in polyaxonal pockets (FIG. 5D). In contrast, distal segments of WT nerve had significantly fewer axonal sprouts and fewer polyaxonal pockets (FIGS. 5A and 5C).

It was next determined whether regenerating sprouts associated with the individual Bungner bands. Bungner bands are the denervated Schwann cell bands of the degenerating myelinated fibers of the distal nerve stumps. These Bungner bands could be reliably identified for the first week after nerve crush by the presence of myelin debris within the Schwann cells (Stoll et al., J. Neurocytol. 18:671-83 (1989); Schäfer et al., Neuron 6:1107-13 (1996); and Griffin et al., Exp. Neurol. 223:60-71 (2010)). The numbers of axonal sprouts/Bungner band was greater in the BACE1 KO nerves than in WT nerves (FIG. 5) at 5 days post crush. The increase in sprouts/Schwann cell was true both of the degenerating myelinated fibers, identified by their myelin debris, and of small regenerating fibers without myelin debris, many of which represent regenerated Remak bundles (FIG. 5C). Thus, initial axonal growth is accelerated in BACE1 KO, and the number of sprouts/Bungner band is greater in injured nerves of BACE1 KO mice.

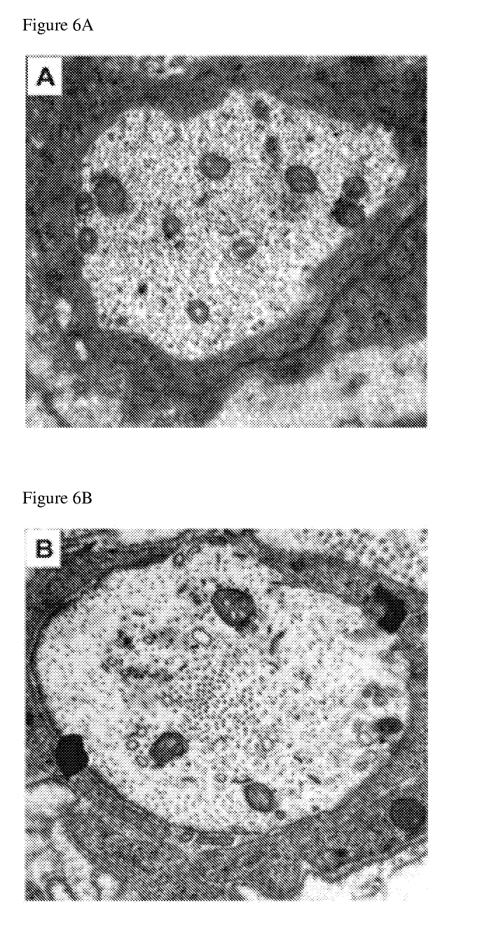

A second ultrastructural feature in some of the BACE1 KO nerves was the prominence of microtubules in the axons. In a minority of premyelinating axons the change was extreme, with many microtubules collected into a central or paracentral “channel” (FIG. 6). In such fibers, neurofilaments were largely within a peripheral ring of axoplasm. The segregation was less dramatic in most BACE1 KO fibers, but there were clusters of microtubules scattered throughout the axoplasm.

Next, axonal regeneration was quantitated after crush injury in both the spatial and temporal patterns of sprout invasion into the distal stump. At defined times, more sprouts were found at each level of the distal stump. At defined levels, earlier and faster increases in sprout numbers were observed with time. As an example of the distribution at a specific time, there were more axons with diameters >2 μm in transverse sections taken both 4 and 8 mm away from the crush site in BACE1 KO than in WT littermate nerves 10 days post-crush. As an example of the temporal differences, regenerating axons with diameters >2 μm in plastic nerves of BACE1 KO and control littermate at 10 and 15 days post crush were quanitified in the segment 6-8 mm distal to the crush site. The BACE1 KO nerves had on average 42.0% more axons at 10 days and 35.0% more axons at 15 days than WT littermate nerves (FIG. 7). At each time, the axons were larger in the BACE1 KO nerves than in the control nerves, consistent with their earlier arrival. Furthermore, distal nerve segments 10-12 mm away from crush site were stained with antibody against phosphorylated neurofilament (NF160). BACE1 KO nerve had many more neurofilament-positive axons than were present in WT littermate nerves (FIG. 7). Therefore, lack of BACE1 increases both the numbers of early sprouts/Bungner band and the total number of myelinating axons in the distal stump. These factors act together to produce larger, more mature axons at every given post-crush interval and at each given level of BACE1 KO nerve.

### Example 4

### Faster Reinnervation of Target Muscles in BACE1 KO Mice

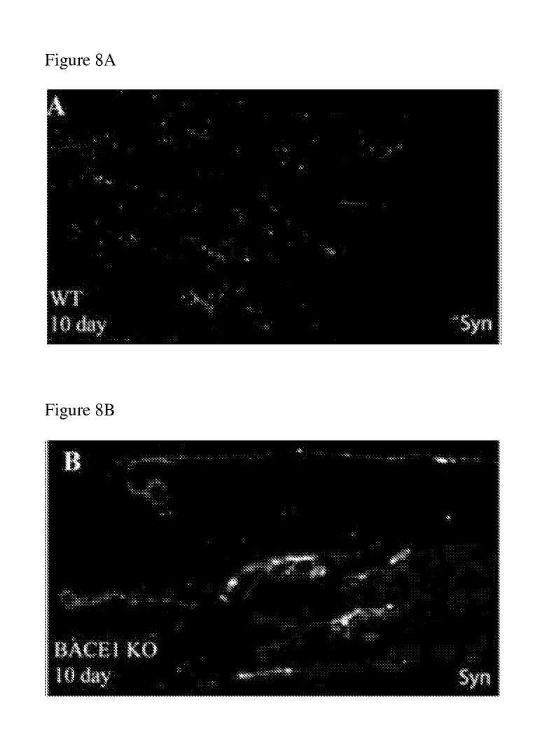

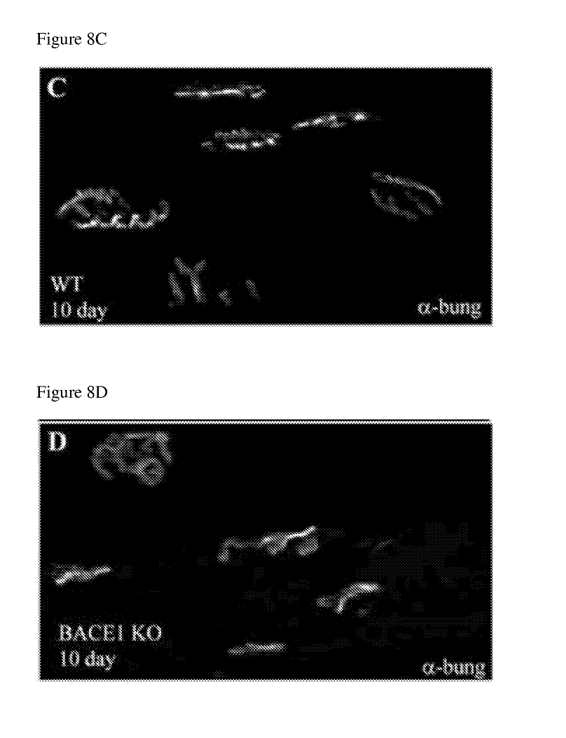

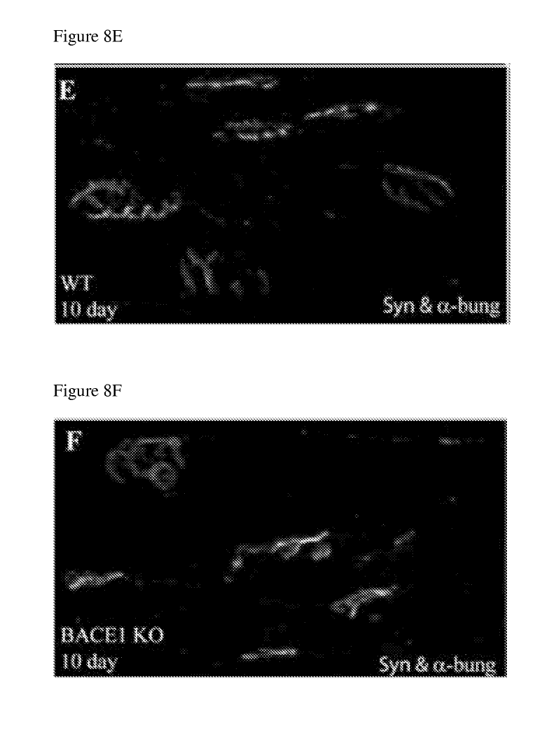

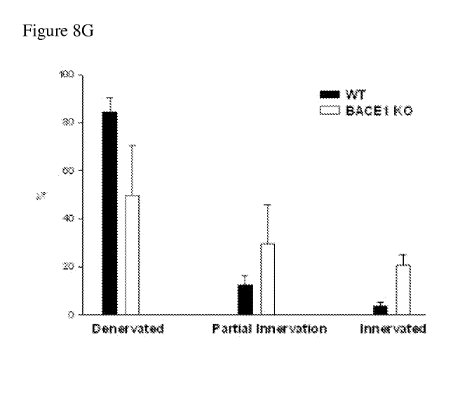

To investigate whether the robust axonal regeneration in BACE1 KO mice leads to accelerated reinnervation of the neuromuscular junctions, the sciatic nerves of BACE1 KO and WT littermate mice were crushed. At 10 days post-crush, BACE1 KO muscles showed significantly improved reinnervation over WT muscles (FIG. 8). Muscle re-innervation in the foot was also examined. Mature pre-synaptic endings were observed as indicated by synaptophysin staining in BACE1 KO compared to WT littermate feet (data not shown), suggesting that reinnervation in the foot occurs faster after crush injury in BACE1 KO mice than WT. These observations demonstrate faster axonal regeneration in BACE1 KO nerves results in faster target reinnervation.

### Example 5

### Improved Debris Clearance and Increased Axonal Regeneration Using Two Structurally Distinct Small Molecule BACE1 Inhibitors

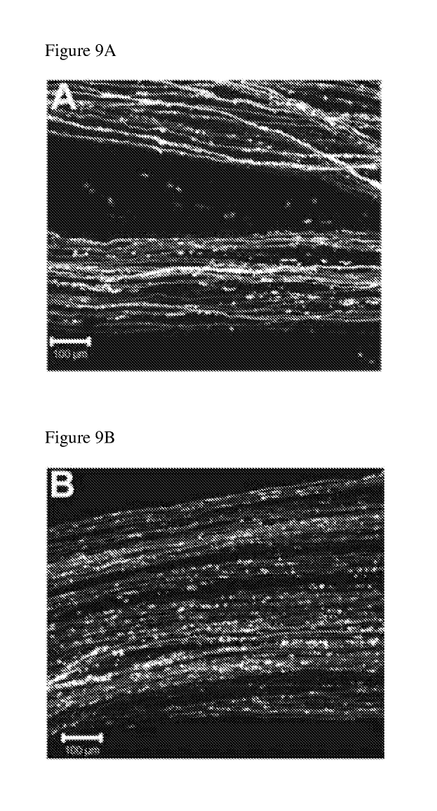

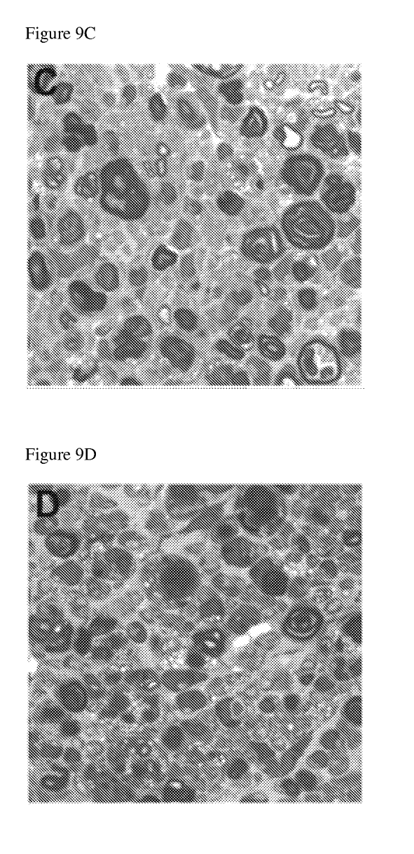

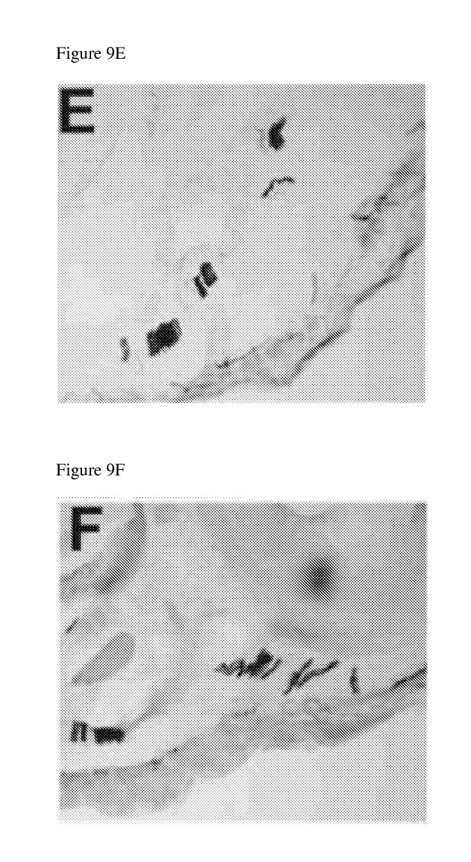

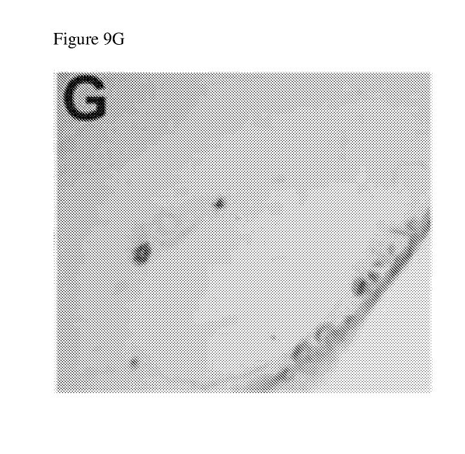

Next, pharmacological BACE1 inhibitors were evaluated for their ability to reproduce the degeneration/regeneration phenotypes observed in injured BACE1 KO nerves. Two structurally different BACE1 inhibitors were used: BACE1 inhibitor IV (Stachel et al., J. Med. Chem. 47:6447-50 (2004)) and WAY 258131 (Malamas et al., J. Med. Chem. 53:1146-58 (2010)). The inhibitors were systemically administered via subcutaneous catheters from Alzet pumps implanted in the interscapular space for 7 or 15 days. At 7 days post-crush, the inhibitor-treated nerves had improved axonal and myelin debris clearance and had significantly more regenerating axons than vehicle-treated mice (FIG. 9). In addition, BACE1 inhibitors increased presynaptic endings in the feet of treated mice as revealed by synaptophysin staining at 2 weeks post-crush (data not shown). Therefore, pharmacological inhibition of BACE1 resulted in degeneration/regeneration phenotypes similar to that observed in BACE1 KO mice.

### Example 6

### Enhanced Regeneration of BACE1KO Axons Through WT Nerve Grafts

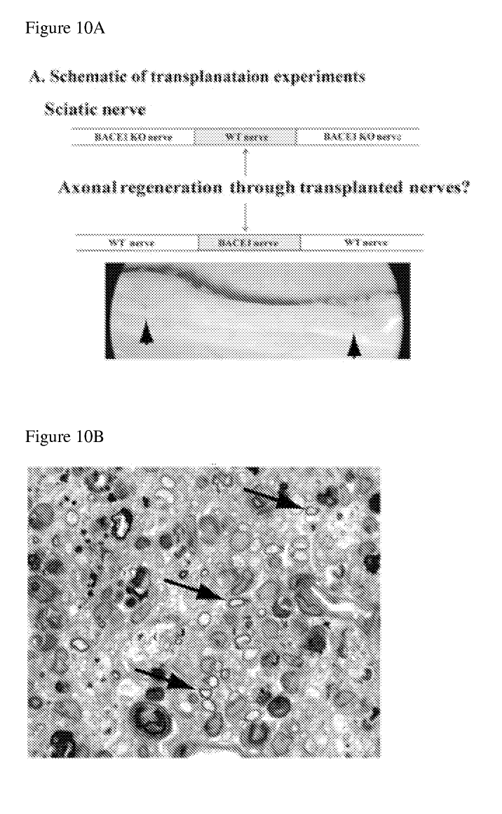

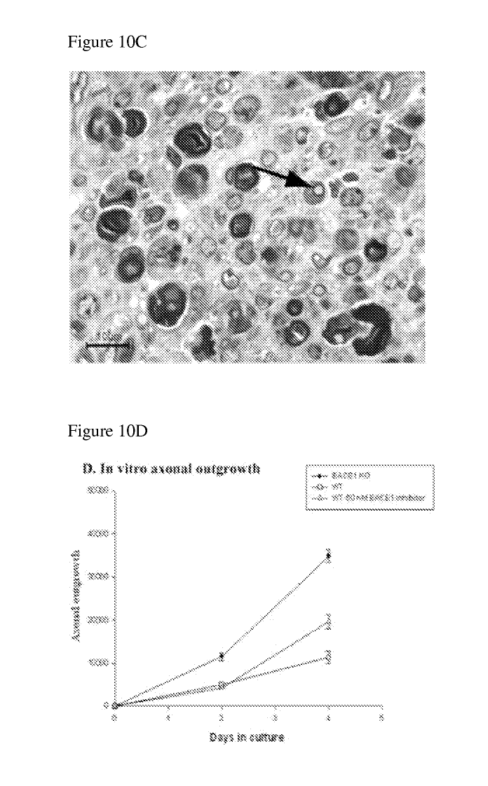

Next, it was determined whether accelerated axonal regeneration in injured BACE1 KO nerves was due to lack of BACE1 activity in the resident cells of the nerve, such as denervated Schwann cells. Normally in the PNS, BACE1 is predominately a neuronal protein, but BACE1 expression and activity are upregulated in non-neuronal cells under non-physiological conditions such as injury and stress (Tamagno et al., J. Neurochem. 104:683-95 (2008)). To examine the cellular basis of accelerated axonal regeneration, reciprocal transplantation of nerve segments was performed between WT and BACE1 KO as diagrammed in FIG. 10A. The growth of BACE1 KO axons was significantly accelerated through a wild-type nerve graft, compared to that of wild-type axons growing through BACE1 KO nerve grafts (FIGS. 10B and 10C). These results excluded BACE1 expression in Schwann cells of the distal stump as being responsible for accelerated regeneration. Thus, either BACE1KO neurons or hematogenous macrophages, or both, could be contributors.

### Example 7

### Accelerated Axonal Outgrowth in DRGs From BACE KO Mice or DRGs Treated with BACE1 Inhibitors

Next, it was determined if lack of neuronal BACE1 accelerates axonal outgrowth in vitro in explants of dorsal root ganglia (DRG). Postnatal (P4-5) primary mouse DRG from WT littermate and BACE1 KO pups were established. Axonal outgrowth at 2 and 4 days post culture were measured. The axons of BACE1 KO DRG explants were on average 119% and 150% longer than those of WT DRG explants at 2 and 4 days post culture, respectively (FIG. 10D). The effect of BACE1 inhibitor on axonal outgrowth of wild-type DRG explants was also investigated (FIG. 10D). 50 nM of BACE1 inhibitor IV (IC₅₀=29 nM) increased axonal outgrowth of WT DRG explants by 98.0% at 4 days post culture. Together, the in vivo transplantation and in vitro data indicate that reducing, by either genetic deletion or pharmacological compounds, of neuronal BACE1 accelerates axonal outgrowth.

### Discussion

As described above, reduced BACE1 activity accelerates the rate of axonal regeneration after nerve crush. The data suggest two components of the mechanism. First, the enhancement in regeneration is due to speeding of axonal growth rate, rather than to reducing the latency period before growth. Second, in other models, both intrinsic neuronal mechanisms and removal of growth-inhibiting external cues such as myelin debris can contribute to enhanced regeneration. BACE1 inhibition appears to work through both of these pathways.

The distinction between axonal growth rate versus time required for neurons to enter an axonal growth state is a fundamental issue. Many previous reports of improved nerve regeneration have depended on shortening of the latency before axonal growth occurs. The classical demonstration of reduced latency is the “conditioning” model: nerves subjected to a previous axotomy undergo neuronal responses that place them into a “growth state”, so that a second crush of that nerve results in prompt initiation of outgrowth, skipping the 2 day delay seen in naïve animals. The neuronal growth state depends on the expression of growth-related genes such as ATF3 and can be mimicked by transgenic expression of these genes (Seijffers et al., J. Neurosci. 27: 7911-79 (2007)). As described above, it has been discovered that the rate of growth rather than the latency to growth was predominantly affected when using multiple complementary techniques. These findings demonstrate that a strategy of combined approaches is a useful approach for treating nerve injury

Neuronal Effects

The early stages of Wallerian degeneration were comparable in BACE1 KO and control littermate mice after axotomy, and no axonal protection effect was observed from genetic deletion of BACE1 when mice were treated with the axonotoxic compounds paclitaxel or acrylamide. These results contrast with results in embryonic neurons deprived of nerve growth factor (NGF). In NGF withdrawal, axonal degeneration was found to depend on an N-APP fragment. This fragment is generated and acts through a newly identified pathway based on 4 molecules, BACE1, APP, death receptor 6, and caspase 6 (Nikolaev et al., 2009). These data suggest that the N-APP fragment negatively affects axonal health. However, the data described herein demonstrate that the mechanism of axonal degeneration in Wallerian degeneration and following exogenous toxins is different from the effect of growth factor withdrawal documented by Nikolaev et al., 2009. Consistent with the results described herein, Vohra et al. J. Neurosci. 30:13729-38 (2010) has reported that axonal degeneration caused by mechanical insult or vincristine intoxication occurs independent of APP cleavage and caspase 6 activation in cultured neurons.

In BACE1 KO axons, the presumed neuronal contribution is evident in cultured neurons, e.g., the BACE1-null neurites grow at faster rates. The faster outgrowth could correlate with alterations in the organization of the axonal cytoskeleton. This is supported by the presence of prominent channels of microtubules in BACE1 KO sprouts in vivo. Increased microtubule stability produced by low concentrations of Taxol® results in faster axonal outgrowth (Ertürk et al., J. Neurosci. 34:9169-80 (2007)). At any given interval after nerve crush, more total regenerating axons was observed in crushed BACE1 KO nerves compared to WT littermate nerves. In addition, more sprouts/ Schwann cell band was observed at early stages in BACE1 KO nerve. That this difference correlates with better regeneration is perhaps surprising, but it suggests that increased sprout numbers may increase the capacity to explore for optimal growth environments in the distal stump, e.g., allowing more “shots on goal” for regenerating sprouts.

Effect on Debris Clearance

Genetic deletion of BACE1 resulted in faster clearance of debris from degenerated fibers in the distal stump. BACE1 KO PNS nerves are known to be hypomyelinated (Willem et al., Science 314:664-6 (2006); and Hu et al., Nat. Neurosci. 12:1520-5 (2006)) a difference that is due to reduced neuregulin 1 signaling. The faster clearance of myelin debris from the BACE1 KO mice is likely due in part to less myelin at baseline. However, as faster clearance of axonal debris was observed in BACE1 KO nerves, the results are reflective of an enhanced general debris clearance mechanism. Consistent with this observation, short-term pharmacological inhibition of BACE1 resulted in faster debris clearance from WT nerves undergoing Wallerian degeneration. The numbers of macrophages in WT and BACE1 KO nerves were similar at early time points after injury, but the BACE1 KO macrophages appear more effective in phagocytosis.

Because elimination of BACE1 contributes to more efficient phagocytosis both in vivo and in vitro, the data indicates that there is likely a substrate for BACE1 in macrophages. BACE1 cleaves galactoside 2,6-sialyltransferase I (ST6Gal-I), a transmembrane protein expressed by macrophages (Kitazume et al., Proc. Natl. Acad. Sci. USA 98:13554-13559 (2001); and Woodard-Grice et al., J. Biol. Chem. 283:26364-73 (2008)). ST6Gal-1 could be a substrate of BACE1 that leads to more rapid phagocytosis when it is not cleaved by BACE1. Another attractive known contributor to phagocytosis is the triggering receptor expressed on macrophage 2 (TREM2). Increasing TREM2 expression on macrophages has been shown to speed phagocytosis of myelin in experimental allergic encephalomyelitis (Takahashi et al., PLoS Med. 4(4):e124 (2007)). TREM2 on the membrane of macrophages is believed to be the active form and cell surface TREM2 is associated with phagocytosis (Turnbull et al., J. Immunol. 177:3520-4 (2006); Piccio et al., Eur. J. Immunol. 37:1290-1301 (2007); and Hsieh et al., J. Neurochem. 109:1144-56 (2009)). More full length TREM2 on the surface of macrophages could drive phagocytosis, and BACE1 inhibition leads directly or indirectly to more TREM2 on cell membrane. A third candidate is the known BACE1 substrate, P-selectin glycoprotein ligand-1 (PSGL-1) (Lichtenthaler et al., J. Biol. Chem. 278:48713-9 (2003)), although the BACE1-cleaved fragment of PSGL-1 is currently thought to play a role in recruitment of leukocytes from the blood to the tissue (Lichtenthaler et al., 2003; and McEver and Cummings, J. Clin. Invest. 100:S97-103 (1997)).

As described above, cultured BACE1 KO macrophages are more efficient in in vitro phagocytosis activity of IgG-coated beads, a capacity dependent on macrophage Fc receptor. Recent data implicated natural IgG and IgM antibodies to peripheral nerve components in accelerating clearance of myelin debris during Wallerian degeneration (Vargas et al., 2010). Thus, BACE1 inhibition may alter Fc-mediated phagocytosis.

BACE1 in Schwann Cells

Denervated Schwann cells have BACE1 activity (data not shown). However, two lines of data argue against Schwann cells being responsible for the observed faster axonal growth. First, 15 days after transplantation of WT nerve grafts into BACE1 KO recipient sciatic nerves there was more extensive regeneration than when WT or BACE1 KO grafts were transplanted into WT recipient nerves. This indicates that the major component of the effect on regeneration is contributed by recipient cells (neuronal or macrophage) rather than by donor Schwann cells. The second line of evidence is the greater rate of outgrowth that was seen in cultured DRG neurons, especially between 2 and 4 days after explantation. These cultures were from P4-5 mice, to make them as consonant as possible with the adults used in the in vivo studies. This evidence suggests that at least part of the “recipient” effect is neuronal, because there are few macrophages in these cultures.

Effects on Axonal Outgrowth

Several complementary assays were used to assess in vivo axonal regeneration in crushed nerves, including the distribution of GAP-43 protein and neurobiotin-labeling. These two measures helped separate an effect on latency from that of enhanced outgrowth of regenerating sprouts. Using these techniques, it was found that most axons had not started growing in either the WT littermates or in the BACE1 KO animals two days after nerve crush. Rather, the axons had retracted or degenerated back from the site of the crush slightly, with the largest fibers more affected than the smaller fibers, as previously described in wild type animals (Ramon y Cajal, 1913). However, greater penetration into the distal stump was present in the BACE1 KO animals than in wild-type animals by day 3, and this difference increased over the next 4 days (6-7 days post-crush is the longest interval after axotomy that it is possible to “catch” the fastest growing fibers within the sciatic nerve of the mouse). After this time, a high proportion of the sprouts have grown beyond the available length of nerve in both the BACE1 KO and littermate animals.

No to little axonal regeneration in nerves of BACE1 KO and WT littermate mice were observed 2 days after crush, indicating that BACE1 KO animals are not in a neuronal “growth state” of the type produced by earlier axotomy in the “conditioning lesion” (Lanners and Grafstein, Brain Res. 196:547-53 (1980); McQuarrie, J. Comp. Neurol. 231:239-49 (1985); Jacob et al., J. Neurobiol. 24:356-67 (1993); and Seijffers et al., J. Neurosci. 27: 7911-7920 (2007)) before the crush. In peripheral nerves, a persistent axotomy-like growth state has been achieved by the genetically engineered persistent expression of some axotomy-related “genes” such as the transcription factors ATF3 (Seijffers et al., 2007). Rather, the growth state needs to be induced by axotomy in the BACE1 KO as it does in wild-type animals. The BACE1 KO axons grew faster than WT once the 2 day latency period had passed. In short nerves, such as those of the rodents, reduction in the latency period can substantially reduce the time required for growing fibers to reach their targets. However, in long nerves, such as those in man, the time to reinnervation of a target which is several cm from the site of axotomy would be little affected by elimination of the latency period. Under these circumstances, a faster rate of outgrowth would have a substantial effect.

The results described herein demonstrate that faster rate of outgrowth associated with BACE1 inhibition will be useful in speeding nerve regeneration in human conditions. In man, axonal regeneration is sufficiently slow such that denervated Schwann cells, which provide a permissive micro-environment for regeneration, and target tissues are both at risk for undergoing atrophy and death, precluding functional recovery (Hoke, Nat. Clin. Pract. Neurol. 2:448-54 (2006); Gordon et al., Neurosurgery 5:A132-44 (2009); and Griffin et al., Exp. Neurol. 223:60-71 (2010)). This situation underscores the critical need for agents, such as BACE1 inhibitors, that potentially can speed up axonal regeneration. BACE1 has been an attractive drug target for Alzheimer's disease. Over the past 10 years, numerous BACE1 inhibitors have been developed (reviewed in Citron, Trends Pharmacol. Sci. 25:92-7 (2004); and Silvestri, Med. Res. Rev. 29:295-338 (2008), which are hereby incorporated by reference). Although most, if not all, of these inhibitors do not cross the blood-brain barrier well, existing BACE1 inhibitors can successfully access the PNS because the barriers are more promiscuous in the PNS than brain. In addition, the blood-nerve barrier is broken down in the distal stump of injured nerves (George and Griffin, Exp. Neurol. 129:225-36 (1994); and Vargas and Barres, Annu. Rev. Neurosci. 30:153-79 (2007)) making the drug more assessable. Accordingly, inhibition of BACE1 is a novel molecular mechanism and BACE1 inhibitors are novel therapeutics for treating nerve injury.

The results reported herein were obtained using the following methods and materials.

### Animals

BACE1 knockout (KO) mice and control wild-type (WT) littermates were on a mixed 129/BL6 line and for paclitaxel treatment on a C57 background, as previously described (Cai et al., Nat. Neurosci. 4:233-4 (2001); and Savonenko et al., Proc. Natl. Acad. Sci. USA 105:5585-90 (2008)). For a subset of experiments, BACE1 KO mice were crossed to mice expressing yellow fluorescent protein (YFP) driven by the thy1.2 neuronal promoter (line YFP-H) (Feng et al., Neuron 28:41-51 (2000)). In these animals, a small proportion (3-10%) of neurons in the ventral horn and dorsal root ganglia express YFP. YFP-positive axons were examined in the sciatic nerves of BACE1 KO-YFP mice and WT-YFP littermates.

A total of 220 mice (8-12 week old) were used in the above-described examples. In each experiment, roughly equal numbers of females and males were used and no bias was observed. The animal surgeries and experimental protocols were approved by the Animal Care and Use Committee of the Johns Hopkins University School of Medicine.

### Sciatic Nerve Injury

Mice were deeply anesthetized with isoflurane and left sciatic nerves in the mid thigh or at the sciatic notch level were exposed and crushed by pulling tight a loop of 8-0 nylon black sutures for 30-40 seconds. The crush sites were marked with a 10-0 nylon black suture going through the epineurium only. For sciatic nerve transection, a small ˜2 mm piece of nerve was dissected out and the two nerve stumps were turned away from each other to prevent axonal regeneration to distal stumps. The distal stumps and contralateral nerves were collected 1-15 days post-transection.

### Paclitaxel Administration

Paclitaxel (LC laboratories) was dissolved in 100% ethonal. Equal volumes of paclitaxel solution and cremophor (Sigma-Aldrich, St. Louis, Mo.) were vigorously vortexed for 10 minutes. Ice cold saline (80% of the final volume) was added to freshly made paclitaxel/cremophor solution immediately before injection. 30 mg/kg body weight of paclitaxel in this solution was injected intravenously via tail vein at 3 times a week for 2 weeks. Mice (n=3 per genotype) had no significant weight loss.

### Acrylamide Administration

Acrylamide (Sigma-Aldrich, St. Louis, Mo.) was dissolved in drinking water at 250 ppm (parts per million). Animal were intoxicated with acrylamide for 4 weeks. Sciatic nerves and hind feet were harvested from intoxicated WT and BACE1 KO mice (n=4 per genotype) and processed for plastic sectioning or immunohistochemistry as described below.

### Axonal Regeneration in Vivo Assays

Regeneration into the distal stump was assessed by several complementary techniques. The distribution of the axotomy-induced neuronal protein GAP43, a marker that labels the proximal stump and regenerating sprouts of crushed axons and is enriched in the growth cones, was analyzed. In longitudinal sections of the crushed nerves, the distribution of two other axonally transported markers—neurobiotin and the neuronally expressed yellow fluorescent protein (YFP) (Feng et al., 2000) was also assessed within the distal stump. In addition, electron microscopy of transverse sections of the nerve was performed at defined intervals after axotomy. Finally, the time required for axons of different classes to reach and reinnervate their targets was measured. These techniques were used to separately assess the latency to the onset of regeneration, the rate of outgrowth, and the time to reinnervation of targets. Technical details of each method are described below.

### Neurobiotin Labeling

In a subset of experiments, exogenous tracer neurobiotin (Vector Laboratories, Burlingame, Calif.) that is rapidly transported intraaxonally was used. This method labels axons, including sprouts of regenerating axons entering the distal stump (Lapper and Bolam, J. Neurosci. Methods 39:163174 (1991); and Jacquin et al., J. Neurosci. Methods 45:71-86 (1992)). Nerves were first crushed as described above, and then 2.5% neurobiotin was injected via glass micropipettes (25 μm tip) into nerves proximal to the original crush sites at 5 days later. Three to four hours later, animals were perfused as detailed below and nerves were collected. Neurobiotin transported along regenerating axons was detected histochemically.

### Sciatic Nerve Transplantations

Reciprocal transplantation of WT nerve segments into BACE1 KO recipients and of BACE1 KO segments into WT recipients were performed. In each transplantation, a 10 mm nerve segment from the donor nerve was removed. The left sciatic nerve of each mouse recipient was transected and the donor nerve segment was promptly transplanted into the host sciatic nerve and sutured in place with 10-0 nylon in proximal-distal orientation. Two weeks later, the nerves were collected and 2 mm segments in the middle of donor nerves and host nerves were processed for examination of axonal regeneration in transverse plastic sections.

Implanting of Alzet Pumps for Infusion of BACE1 Inhibitor

Alzet pumps were filled, primed, and implanted as previously described in Farah, J. Neurosci. Methods 134:169-178 (2004); and Farah and Easter, J. Comp. Neurol. 489:120-134 (2005). Briefly, Alzet pumps (Model 2001 of Alzet osmotic pumps, Durect Corporation, Palo Alto, Calif.), were filled with 200-220 μl of BACE1 inhibitor IV (Calbiochem) at concentrations of 1-4 mg/ml or with WAY 258131 (Malamas et al., J. Med. Chem. 53:1146-58 (2010)) at doses 10-30 mg/ml. The latter compound was synthesized by the NeuroTranslational team at the Brain Science Institute at Johns Hopkins University. Both inhibitors were dissolved in DMSO. According to the manufacturer's data, the pump continuously supplied 1-30 μg/hour of inhibitor for 7 days. To minimize the delay between implantation and the onset of release, the filled pumps were incubated in 1×PBS at 37° C. for 4 hours immediately before implantation. The mice were anesthetized by inhalation of isoflurane, and an incision, about 1.5 cm long, was made in the skin on top of the interscapular space. A filled Alzet pump was implanted subcutaneously into each mouse and incisions were closed with 9 mm stainless steel wound clips.

### Tissue Processing

For fluorescent microscopy and immunostaining, mice were deeply anesthetized with 10% chloral hydrate and killed by transcardial perfusion with 1×PBS followed by 2% paraformaldehyde in 0.1 mM phosphate buffer, pH 7.4, for 10-15 minutes. Tissues were then postfixed in the same fixative for 4-16 hr at 4° C. Sciatic nerves and gastrocnemius muscles were cryoprotected in 30% sucrose in 1×PBS overnight and quickly frozen in cold 2-methylbutane. Tissues were frozen sectioned on a cryostat at thickness of 10 or 20 μm for sciatic nerves and 50 μm for gastrocnemius muscles. In addition, the hind feet were decalcified, cryoprotected in 30% sucrose in 1×PBS, and sectioned at 50 μm thickness. Sciatic nerves were sectioned longitudinally using a Leica cryostat (model CM3050S, Leica Microsystems, Buffalo Grove, Ill.). Gastrocnemius muscles and foot were cross-sectioned by a freezing sliding microtome (Microm HM 450, Thermo Scientific, Waltham, Mass.).

To visualize YFP-positive degenerating and regenerating axons, animals were perfused as described above and sciatic nerves were collected. Whole-mounted sciatic nerves were imaged under a confocal microscope and Z-stack images were collected through the depth of the nerves.

For immunocytochemistry, nonspecific antibody binding was blocked by 5% goat serum/0.3% triton (Sigma) in 1×PBS for 1-2 hrs at room temperature. Sections were then incubated with primary antibodies (table 1) overnight at 4° C. After 3×PBS washes, sections were incubated with appropriate secondary antibodies at room temperature for 1 hour, washed 3× in PBS, mounted (Prolong Antifade Kit, Invitrogen, Carlsbad, Calif.), and coverslipped. Appropriate secondary, anti-mouse, rat, or rabbit antibodies conjugated with Alexa fluor 488 or 647 (Invitrogen, Carlsbad, Calif.) were used. For immunoperoxidase labeling, sections were treated with 0.3% H₂0₂ before staining to quench activity of endogenous peroxidase. Primary antibodies were detected with ABC kit. Images of immunostained tissues were acquired by either standard light microscope, laser scanning confocal microscope (Model LSM 510, Carl Zeiss), or Axio Imager with an Apotome (Carl Zeiss).

For ultrastructural analyses, nerves were postfixed in OsO₄ and embedded in Epon. Cross sections, at 1 μm thickness, were stained for toluidine blue and examined under light microscopy with 100× oil-immersion objective. 70 nm thin sections were obtained and stained for citrate/uranyl acetate. Electron micrographs were acquired using a Hitachi 7600 or Zeiss Libra transmission electron microscope.

### Quantification

100 degenerating fibers per genotype (50 per nerve) were identified using an electron microscope. The degenerating fibers were systematically chosen by scanning the EM section in lines from left to right and imaging fibers at 8,000-12,000×. For each degenerating myelinated fiber (defined by having degenerating myelin within a single basal lamina), the number of axonal sprouts growing beneath the basal lamina were counted, and thus these were axonal sprouts per Bungner's band.

At the light microscopic level, regenerating axons were counted at 10 and 15 days post-crush in 1 μm toluidine blue-stained transverse plastic sections taken 6-8 mm distal to the crush site. Axons were counted in each of 4 fields of view at 100× that were randomly chosen for each nerve (n=3 for each WT and BACE1 KO nerves). A total number of regenerating axons per nerve was calculated by multiplying the average density of axons in the sampled area of each nerve by the cross sectional area of that nerve.

Reinnervation of neuromuscular junctions was examined in WT and BACE1 KO gastrocnemius muscles 10 days after sciatic nerve crush at mid thigh level. Muscles sections (at 50 μm thickness) were stained for synaptophysin (to mark presynaptic terminals) and α-bungarotoxin (to mark postsynaptic receptors). 200 neuromuscular junctions per genotype (n=3 animal per genotype) were scored as denervated (no overlap of pre- and post-synaptic staining), partially reinnervated (some overlap of pre- and post-synaptic staining), or fully reinnervated (overlap of pre- and post-synaptic staining comparable to un-operated nerves).

### Dorsal Root Ganglia Explants

Postnatal day 4-5 dorsal root ganglia (DRG) were dissected out of WT and BACE1 KO mice, and were maintained in neurobasal medium containing 2% B27 supplement and 50 ng/ml NGF (all reagents from Invitrogen, Carlsbad, Calif.). In some of the WT DRG explants, 50 nM-1 μM BACE1 inhibitor IV was added daily to the media. At least 8 explants were used for each condition. Axonal outgrowth of the DRG was monitored daily under phase-contrast microscopy, and images were acquired with Openlab software at 2 and 4 days post culture. The axon lengths were measured from the borders of the explants borders to the tips of axons using ImageJ (http://rsb.info.nih.gov/ij/). An average of all axonal lengths for each explant was then determined as described previously (Nguyen et al., J. Neurosci. 29:630-637 (2009)).

### Macrophage Culturing and in Vitro Phagocytosis

Mice (8-10 weeks old) were euthanized by CO₂ inhalation. Peritoneal cells were extracted by injection of RPMI-1640 media plus 10% FCS (both from Invitrogen, Carlsbad, Calif.) into the peritoneal cavity, followed immediately by withdrawal. Cells were plated on coverslips in the same media for overnight. Polystyrene beads with 2 μm diameters were coated with mouse IgG (5 mg/ml; Equitech Bio, Kerrville, Tex.) for 1 hour at 37° C. Coated beads were centrifuged onto adherent macrophages at a ratio of 10:1 for 1 minute at 50 g. Following 3 or 5 minutes incubation at 37° C., non-ingested beads were washed 3 times with cold 1×PBS and cells were fixed in 3.7% formalin solution. 10 fields of view at 20× were photographed by brightfield, and beads ingested by each cell were counted in 200 macrophages per animal (n=3 per genotype).

### Statistical Analysis

All statistical analyses were performed with Student's t test. SigmaPlot software was used to perform the analyses and any value of p<0.05 was scored as statistically significant. Graphed data are presented as mean±SEM.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### INCORPORATION BY REFERENCE

All patents, publications, and accession numbers mentioned in this specification are herein incorporated by reference to the same extent as if each independent patent, publication, and accession number (e.g., the sequences disclosed therein) was specifically and individually indicated to be incorporated by reference.

1. A method for enhancing axonal outgrowth or the reinnervation of a target tissue, the method comprising contacting an axon with an effective amount of an agent that reduces the expression or biological activity of a β-site amyloid precursor protein cleaving enzyme 1 (BACE1), thereby enhancing axonal outgrowth or the reinnervation of a target tissue.

2-3. (canceled)

4. The method of claim 1, wherein the method accelerates axonal outgrowth.

5. The method of claim 1, wherein the method is carried out in vitro or in vivo.

6. The method of claim 1, wherein the axon is a motor neuron axon.

7. A method of preventing, treating, or reducing symptoms of nerve injury in a subject or of increasing axonal regeneration, the method comprising administering to a subject an effective amount of an agent that reduces the expression or biological activity of a β-site amyloid precursor protein cleaving enzyme 1 (BACE1) thereby preventing, treating or reducing symptoms of nerve injury or increasing axonal regeneration.

8. (canceled)

9. The method of claim 7, wherein the method comprises administering the agent in an amount sufficient to increase nerve regeneration, reinnervate neuromuscular junctions, increase axon regeneration, or increase the formation of new presynaptic terminals in the subject in the subject.

10. (canceled)

11. The method of claim 7, wherein the method comprises administering the agent in an amount sufficient to increase clearance of axon and myelin debris in the subject.

12. The method of claim 7, wherein the nerve injury is a peripheral or central nervous system injury, or a neuropathy characterized by degeneration of axons.

13-14. (canceled)

15. The method of claim 14, wherein the neuropathy is diabetic or chemotherapy-induced neuropathy.

16. (canceled)

17. The method of claim 1, wherein the agent is a peptide, polypeptide, polynucleotide, or small molecule.

18. The method of claim 17, wherein the agent is an inhibitory nucleic acid molecule.

19. The method of claim 18, wherein the inhibitory nucleic acid molecule is an antisense nucleic acid molecule, an siRNA, or an shRNA that reduces the expression of BACE1.

20. The method of claim 1, wherein the agent is an antibody that i) specifically binds to BACE1, or ii) specifically binds to a protein that interacts with BACE1, wherein binding of the antibody to the protein inhibits BACE1 cleavage of amyloid precursor protein.

21. The method of claim 1, wherein the agent is selected from a group consisting of a peptidomimetic inhibitor of BACE1, a non-peptidomimetic inhibitor of BACE1, a macrocyclic inhibitor of BACE1, LY 2811376, BACE1 inhibitor IV, OM99-2, OM00-3, GT-1017, KMI-429, KMI-570, KMI-684, CTS-2116, WAY-258131, AZ29, and a pharmaceutically acceptable salt or prodrug thereof.

22-26. (canceled)

28. A therapeutic method to increase nerve regeneration in a mammalian subject after peripheral nerve injury, wherein the method comprises the steps of:
a. identifying a subject with at least one injury to the peripheral nervous system; and
b. administering a BACE1 inhibitor to the subject in an amount and for a time sufficient to increase regeneration of nerve tissue.

29. The method of claim 28, wherein the method further comprises the steps of:
a. measuring the amount of nerve regeneration in the subject after administration of the BACE1 inhibitor;
b. comparing the amount of nerve regeneration in the subject with a control; and
c. modifying the amount and time of BACE1 administration so that nerve regeneration is increased in the subject as compared to the control.

30-36. (canceled)
